(19) United States
(12) Patent Application Publication
(10) Pub. No.: US 2020/0375642 A1
(43) Pub. Date: Dec. 3, 2020

(54) ALL-IN-ONE ODONTOID FIXATION GUIDE APPARATUS, SYSTEMS, AND METHODS

(71) Applicant: Andreas C. TOMAC, Miami, FL (US)

(72) Inventor: Andreas C. TOMAC, Miami, FL (US)

(21) Appl. No.: 16/424,441

(22) Filed: May 28, 2019

(51) Int. Cl. A61B 17/88

(52) U.S. Cl. CPC A61B 17/8811; A61B 2017/00477; A61B 17/8819; A61B 17/8822

(57) ABSTRACT

An all-in-one odontoid fracture repair apparatus, system, and method may include an odontoid guide comprising an odontoid guide cannula having a proximal end, a distal end, and a first longitudinal passage extending through the odontoid guide cannula between the proximal and distal ends of the odontoid guide. The odontoid guide may include a vertebra coupler located at the distal end of the odontoid guide cannula that may be configured to couple to a first vertebra of a patient. The odontoid guide may be configured to receive bone cement within the first longitudinal passage of the odontoid guide cannula and deliver the bone cement through the first longitudinal passage of the odontoid guide cannula to an odontoid process of the patient in order to repair a fractured or osteoporotic odontoid process in the ever increasing fragile geriatric population.

### TECHNICAL FIELD

The present disclosure relates to surgical devices, systems, and methods. More specifically, the present disclosure relates to improved surgical instruments, systems, and methods for repairing an odontoid fracture in a patient.

### BACKGROUND

Odontoid process fractures are the most common type of fracture experienced in the second cervical vertebral body (i.e., the axis vertebra, or C2) and the most common type of cervical spine fracture in geriatric patients. Odontoid fractures are classified according to their specific fracture pattern as either a type I, II, or Ill odontoid fracture. A type I odontoid fracture extends through the tip of the odontoid process, and is usually stable. A type II odontoid fracture extends across the base of the odontoid process, and is the most common type of odontoid fracture. A type Ill odontoid fracture extends through the vertebral body of C2, and may be stable or unstable.

The incidence of geriatric odontoid fractures is steadily increasing as the population ages. Geriatric odontoid fractures can be very unstable and carry an increased risk for catastrophic neurological events, morbidity, and mortality if they are not promptly stabilized via a suitable surgical procedure. However, surgical techniques for repairing odontoid fractures in geriatric patients are usually complicated by: (1) age related suboptimal bone health; and (2) medical comorbidities within the geriatric population.

Accordingly, it would be desirable to provide surgical instruments, systems, and methods for repairing odontoid fractures in patients that may: (1) provide instant stabilization of an odontoid fracture; (2) promote fusion of the dens to the C2 vertebral body; (3) preserve articulation, range of motion, and mobility of the C1-C2 vertebral joint; and (4) reduce associated risks from medical comorbidities by utilizing an anterior surgical approach.

### SUMMARY

The various systems and methods of the present disclosure have been developed in response to the present state of the art, and in particular, in response to the problems and needs in the art that have not yet been fully solved by currently available surgical instruments, systems, and methods for repairing an odontoid fracture in a patient.

According to some embodiments, an all in one system for odontoid fracture repair may include an odontoid guide fixation system and a bone cement delivery system. The odontoid guide fixation system may include an odontoid guide comprising an odontoid guide cannula having a proximal end and a distal end, a first longitudinal passage extending through the odontoid guide cannula between the proximal and distal ends of the odontoid guide cannula, and a vertebra coupler located at the distal end of the odontoid guide cannula that is configured to couple with a first vertebra of a patient. The bone cement delivery system may be receivable within the first longitudinal passage of the odontoid guide cannula. The bone cement delivery system may include a bone cement retainer comprising a bone cement cannula having a proximal end and a distal end, a second longitudinal passage extending through the bone cement cannula between the proximal and distal ends of the bone cement cannula that is configured to receive bone cement therein, and a bone cement retainer handle coupled to the proximal end of the bone cement cannula that is configured to receive a first force directed proximally. The bone cement delivery system may also include a bone cement ejector comprising a bone cement plunger having a proximal end and a distal end that is configured to be received within the second longitudinal passage of the bone cement cannula, and a bone cement plunger handle coupled to the proximal end of the bone cement plunger that is configured to receive a second force directed distally, in opposition to the first force, to move the bone cement plunger distally with respect to the bone cement cannula and eject the bone cement from the distal end of the bone cement cannula.

In other embodiments, an odontoid fracture repair apparatus may include an odontoid guide comprising an odontoid guide cannula having a proximal end and a distal end, a first longitudinal passage extending through the odontoid guide cannula between the proximal and distal ends of the odontoid guide cannula, and a vertebra coupler located at the distal end of the odontoid guide cannula that is configured to couple to a first vertebra of a patient. The odontoid guide may also be configured to receive a bone cement delivery system within the first longitudinal passage of the odontoid guide cannula.

In yet other embodiments, a method for repairing an odontoid fracture in a patient may include receiving bone cement within a first longitudinal passage of an odontoid guide cannula and delivering the bone cement through the first longitudinal passage of the odontoid guide cannula to an odontoid process of the patient.

These and other features and advantages of the present disclosure will become more fully apparent from the following description and appended claims, or may be learned by the practice of the systems and methods set forth hereinafter.

### BRIEF DESCRIPTION OF THE DRAWINGS

Exemplary embodiments of the disclosure will become more fully apparent from the following description and appended claims, taken in conjunction with the accompanying Figures. Understanding that these Figures depict only exemplary embodiments and are, therefore, not to be considered limiting of the scope of the appended claims, the exemplary embodiments of the present disclosure will be described with additional specificity and detail through use of the accompanying Figures in which:

FIG. 1 is cross sectional side view of a cervical spine 100 of a patient taken along an anterior/posterior plane;

FIG. 2 is a side view of the cervical spine 100 of FIG. 1;

FIG. 3 is a posterior view of the cervical spine 100 of FIG. 1;

FIG. 4 is a posterior/superior view of a portion of the cervical spine 100 of FIG. 1, illustrating an atlas cervical vertebra 110 (C1) coupled to an axis cervical vertebra 120 (C2);

FIG. 5A is a side view of the axis cervical vertebra 120 (C2) of FIG. 4, illustrating an example location for a type II odontoid process fracture;

FIG. 5B is an anterior view of the axis cervical vertebra 120 (C2) of FIG. 4 including a type II odontoid process fracture;

FIG. 6A is a perspective top view of an odontoid guide 200, according to an embodiment of the disclosure;

FIG. 6B is a perspective bottom view of the odontoid guide 200 of FIG. 6A;

FIG. 6C is a right side view of the odontoid guide 200 of FIG. 6A;

FIG. 6D is a left side view of the odontoid guide 200 of FIG. 6A;

FIG. 6E is a distal end view of the odontoid guide 200 of FIG. 6A;

FIG. 6F is a proximal end view of the odontoid guide 200 of FIG. 6A;

FIG. 6G is a close up perspective view of the distal end of the odontoid guide 200 of FIG. 6A;

FIG. 6H is a close up side view of the distal end of the odontoid guide 200 of FIG. 6A;

FIG. 7 is a perspective view of a drill bit 300, according to an embodiment of the disclosure;

FIG. 8 is a perspective view of a drill bit 400, according to another embodiment of the disclosure;

FIG. 9A is a perspective view of a bone cement retainer 500, according to an embodiment of the disclosure;

FIG. 9B is another perspective view of the bone cement retainer 500 of FIG. 9A;

FIG. 10A is a perspective view of a bone cement ejector 600, according to an embodiment of the disclosure;

FIG. 10B is another perspective view of the bone cement ejector 600 of FIG. 10A;

FIG. 11A is a perspective right side view of an adjustable stop member 700, according to an embodiment of the disclosure;

FIG. 11B is a perspective left side view of the adjustable stop member 700 of FIG. 11A;

FIG. 11C is a distal end view of the adjustable stop member 700 of FIG. 11A;

FIG. 12A is a side view of a stop member coupler 800, according to an embodiment of the disclosure;

FIG. 12B is a proximal end view of the stop member coupler 800 of FIG. 12A;

FIG. 12C is a distal end view of the stop member coupler 800 of FIG. 12A;

FIG. 13A is a perspective view of an adapter sleeve 900, according to an embodiment of the disclosure;

FIG. 13B is another perspective view of the adapter sleeve 900 of FIG. 13A;

FIG. 14A is a right side view of a bone screw 1000, according to an embodiment of the disclosure;

FIG. 14B is a left side view of the bone screw 1000 of FIG. 14A;

FIG. 14C is a proximal end view of the bone screw 1000 of FIG. 14A;

FIG. 15A is a perspective view of a bone screwdriver 1100, according to an embodiment of the disclosure;

FIG. 15B is another perspective view of the bone screwdriver 1100 of FIG. 15A;

FIG. 16 is an exploded view of an odontoid fracture repair system 1200, according to an embodiment of the disclosure, where the bone cement retainer 500 and the bone cement ejector 600 may comprise a bone cement delivery system that is receivable within the odontoid guide 200;

FIG. 17A is an exploded view of an odontoid fracture repair system 1300, according to an embodiment of the disclosure, where the bone cement retainer 500 and the bone cement ejector 600 may together comprise a bone cement delivery system that is receivable within the adapter sleeve 900;

FIG. 17B is an exploded view of the odontoid fracture repair system 1300 of FIG. 17A including the drill bit 300, the drill bit 400, the bone screw 1000, and the bone screwdriver 1100;

FIG. 18 illustrates a perspective view of an assembled odontoid fracture repair system 1400, according to an embodiment of the disclosure;

FIG. 19 illustrates a portion of the cross sectional side view of the cervical spine 100 from FIG. 1. More specifically, FIG. 19 depicts a cross sectional side view of portions of the C1, C2, and C3 vertebrae taken along the anterior/posterior plane, including a type II odontoid process fracture 122 formed in the C2 vertebra;

FIG. 20 shows the cervical spine 100 of FIG. 19 with a vertebra coupler 220 of an odontoid guide 200 proximate the C3 vertebra;

FIG. 21 shows the cervical spine 100 of FIG. 19 with the vertebra coupler 220 of FIG. 21 driven into the C3 vertebra;

FIG. 22 shows the cervical spine 100 of FIG. 19 with the drill bit 300 of FIG. 7 projecting from the distal end of the odontoid guide 200 to remove portions of the C2 and C3 vertebrae;

FIG. 23 shows the cervical spine 100 of FIG. 19 with the drill bit 300 removed from the odontoid guide 200 to illustrate the portions of the C2 and C3 vertebrae that have been removed by the drill bit 300;

FIG. 24 shows the cervical spine 100 of FIG. 19 with the drill bit 400 of FIG. 8 projecting from the distal end of the odontoid guide 200 to form bone tunnels through the body of C2 (120) and the fractured odontoid process 123;

FIG. 25 shows the cervical spine 100 of FIG. 19 with the drill bit 400 removed from the odontoid guide 200 to illustrate the bone tunnels 124, 126 formed in C2 and the fractured odontoid process 123, respectively;

FIG. 26 shows the cervical spine 100 of FIG. 19 with the distal end of the bone cement retainer 500 of FIG. 9A projecting from the distal end of the odontoid guide 200 and into the bone tunnels 124, 126 formed in C2 and the fractured odontoid process 123;

FIG. 27 shows the cervical spine 100 of FIG. 19 with bone cement being ejected from the distal end of the bone cement retainer 500;

FIG. 28 shows the cervical spine 100 of FIG. 19 with the bone cement retainer 500 removed from the odontoid guide 200 and bone cement 127 inside the fractured odontoid process 123 and C2;

FIG. 29 shows the cervical spine 100 of FIG. 19 with a bone screw 1000 being inserted into the bone tunnels 124, 126 through the distal end of the odontoid guide 200;

FIG. 30 shows the cervical spine 100 of FIG. 19 with the bone screw 1000 fully inserted through the bone tunnels 124, 126 and being driven into place by the bone screwdriver 1100 to reduce the odontoid fracture 122;

FIG. 31 shows the cervical spine 100 of FIG. 19 after the bone screw 1000 has been fully driven into place by the bone screwdriver 1100, with the bone screwdriver 1100 and the odontoid guide 200 removed; and

FIGS. 32A-32B illustrate a flowchart of a method 1500 for repairing an odontoid fracture in a patient, according to an embodiment of the disclosure.

It is to be understood that the Figures are for purposes of illustrating the concepts of the disclosure and may not be drawn to scale. Furthermore, the Figures illustrate exemplary embodiments and do not represent limitations to the scope of the present disclosure.

### DETAILED DESCRIPTION

Exemplary embodiments of the present disclosure will be best understood by reference to the Figures, wherein like parts are designated by like numerals throughout. It will be readily understood that the components of the present disclosure, as generally described and illustrated in the Figures herein, could be arranged and designed in a wide variety of different configurations. Thus, the following more detailed description of the embodiments of the apparatus, system, and method, as represented in the Figures, is not intended to limit the scope of the present disclosure, as claimed in this or any other application claiming priority to this application, but is merely representative of exemplary embodiments of the present disclosure.

Standard medical directions, planes of reference, and descriptive terminology are employed in this specification. For example, anterior means toward the front of the body. Posterior means toward the back of the body. Superior means toward the head. Inferior means toward the feet. Medial means toward the midline of the body. Lateral means away from the midline of the body. Axial means toward a central axis of the body. Abaxial means away from a central axis of the body. Ipsilateral means on the same side of the body. Contralateral means on the opposite side of the body. A sagittal plane divides a body into right and left portions. A midsagittal plane divides the body into bilaterally symmetric right and left halves. A coronal plane divides a body into anterior and posterior portions. A transverse plane divides a body into superior and inferior portions. These descriptive terms may be applied to an animate or inanimate body.

The phrases “connected to,” “coupled to,” “engaged with,” and “in communication with” refer to any form of interaction between two or more entities, including mechanical, electrical, magnetic, electromagnetic, fluid, and thermal interaction. Two components may be functionally coupled to each other even though they are not in direct contact with each other. The term “abutting” refers to items that are in direct physical contact with each other, although the items may not necessarily be attached together. The phrase “fluid communication” refers to two features that are connected such that a fluid within one feature is able to pass into the other feature.

The word “exemplary” is used herein to mean “serving as an example, instance, or illustration.” Any embodiment described herein as “exemplary” is not necessarily to be construed as preferred or advantageous over other embodiments. While the various aspects of the embodiments are presented in the Figures, the Figures are not necessarily drawn to scale unless specifically indicated.

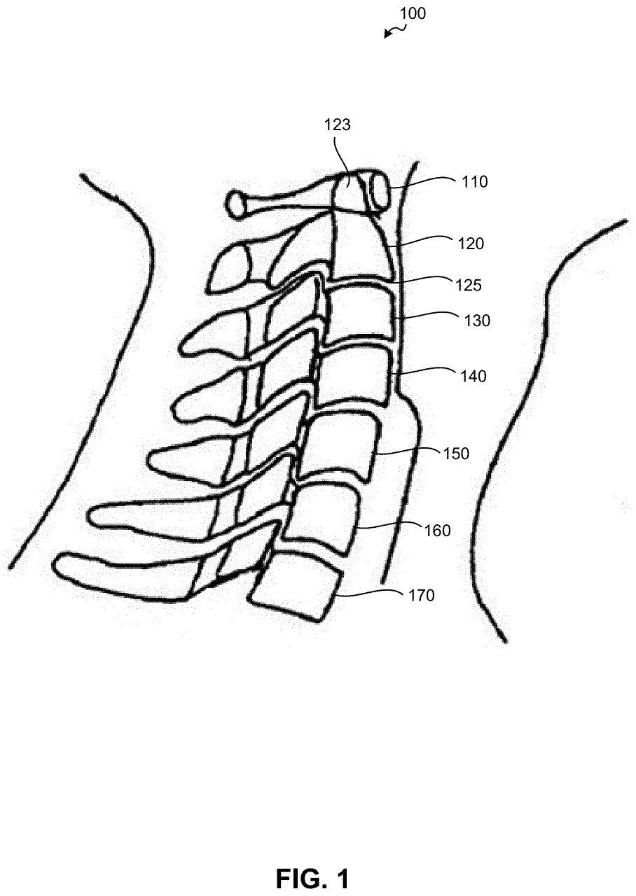

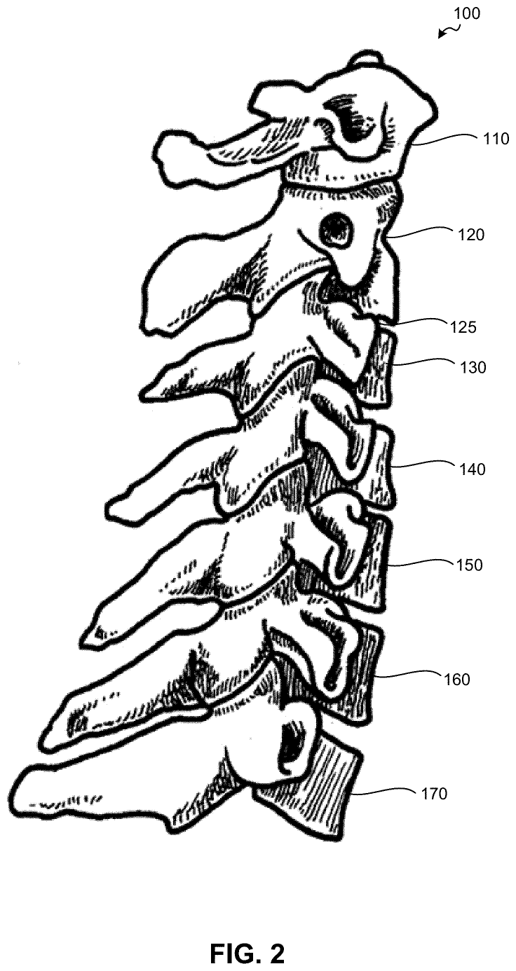

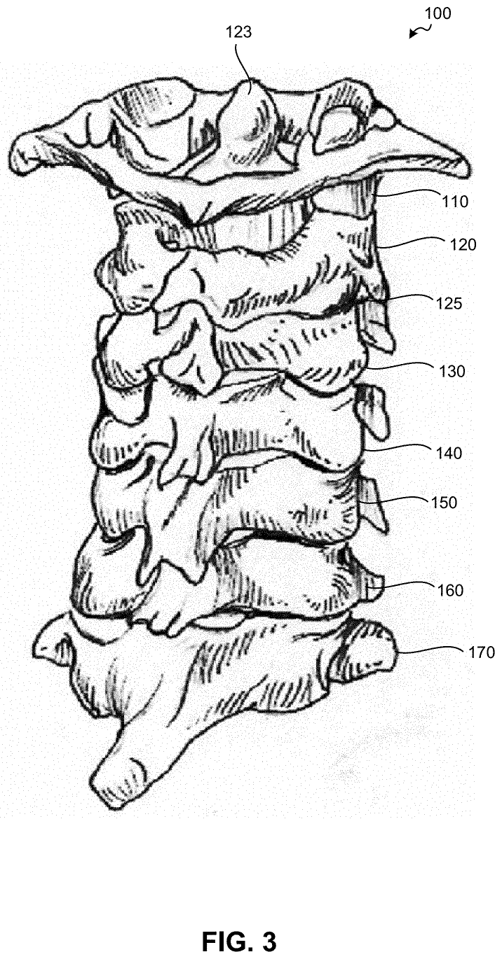

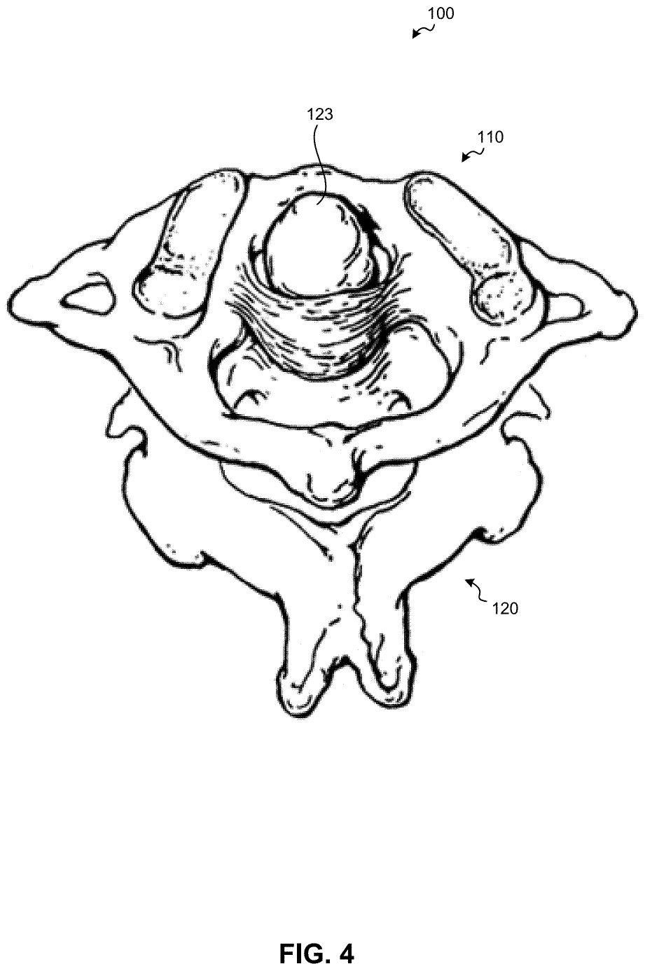

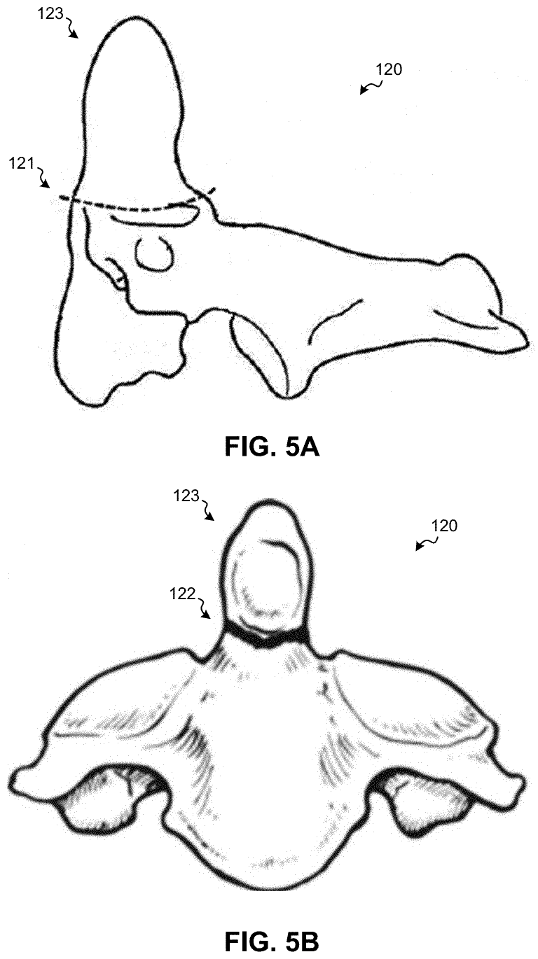

FIGS. 1-5B illustrate various views of cervical spine anatomy, according to examples of the present disclosure. Specifically, FIG. 1 shows a cross sectional side view of a cervical spine 100 of a patient taken along an anterior/posterior plane; FIG. 2 is a side view of the cervical spine 100; FIG. 3 is a posterior view of the cervical spine 100; FIG. 4 is a posterior/superior view of a portion of the cervical spine 100 of FIG. 1 showing an atlas cervical vertebra 110 (C1) coupled to an axis cervical vertebra 120 (C2); FIG. 5A is a side view of the axis cervical vertebra 120 (C2) of FIG. 4 illustrating an example location 121 for a type II odontoid process fracture; and FIG. 5B is an anterior view of the axis cervical vertebra 120 (C2) of FIG. 4, including a type II odontoid process fracture 122. For simplicity, the cervical vertebrae in the present disclosure may be referred to as C1 110, C2 120, C3, 130, C4 140, C5 150, C6 160, and C7 170, respectively. Moreover, the vertebral joint between C2 and C3 may be referred to as the C2-C3 disc 125, and the odontoid process 123 may be simply referred to as the odontoid.

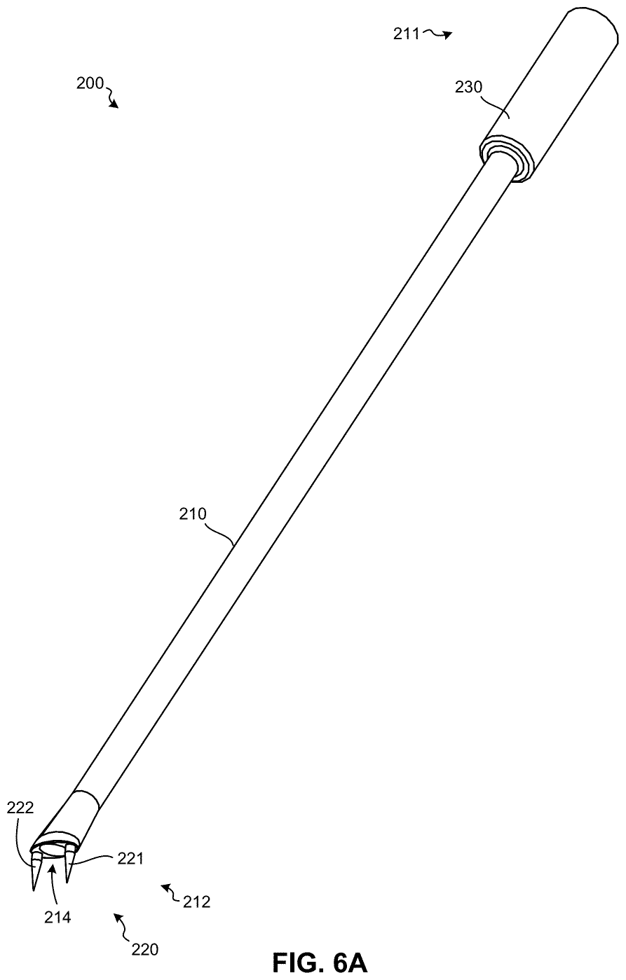

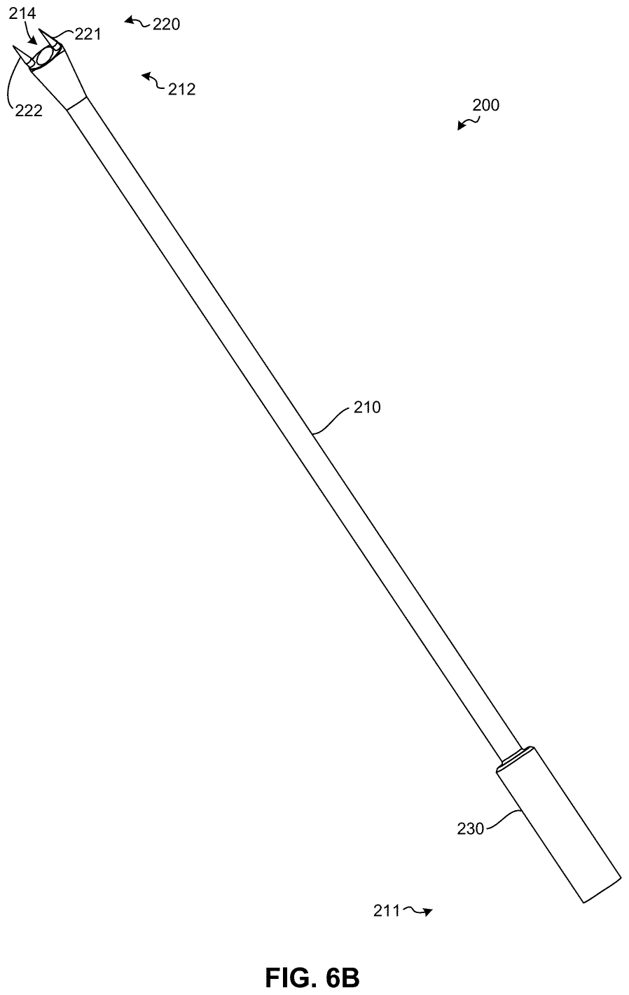

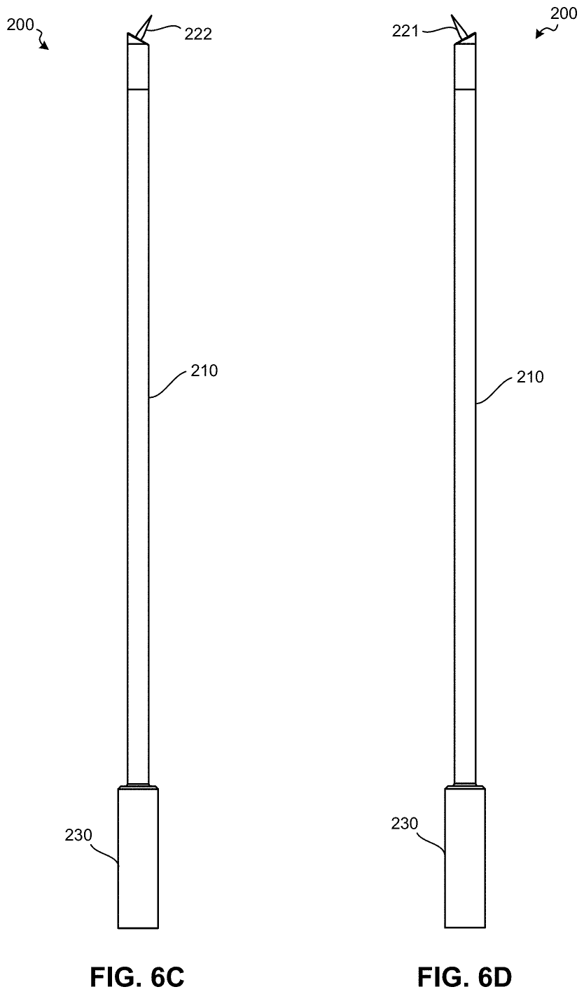

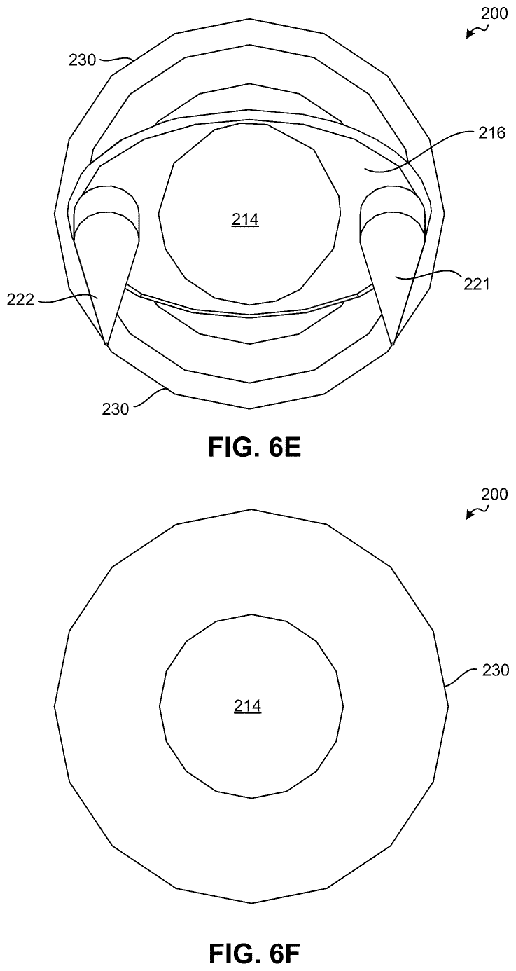

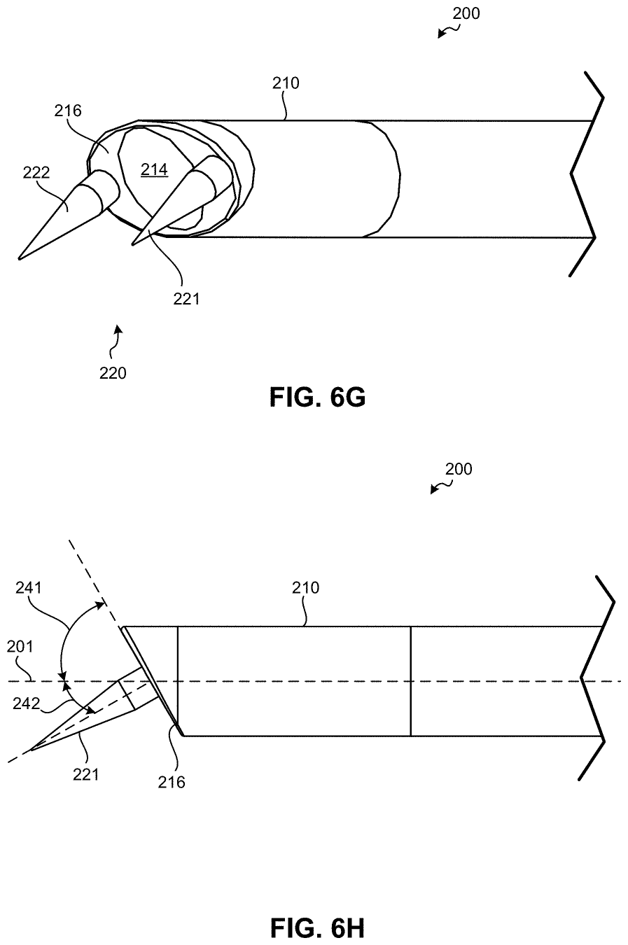

FIGS. 6A-6H illustrate various views of an odontoid guide 200, according to an embodiment of the disclosure. Specifically, FIG. 6A illustrates a perspective top view of the odontoid guide 200; FIG. 6B illustrates a perspective bottom view of the odontoid guide 200; FIG. illustrates a right side view of the odontoid guide 200; FIG. 6D illustrates a left side view of the odontoid guide 200; FIG. 6E illustrates a distal end view of the odontoid guide 200; FIG. 6F illustrates a proximal end view of the odontoid guide 200; FIG. 6G illustrates a close up perspective view of the distal end of the odontoid guide 200; and FIG. 6H illustrates a close up side view of the distal end of the odontoid guide 200. The odontoid guide 200 may generally include an odontoid guide cannula 210, a vertebra coupler 220, and a vertebra manipulator member 230.

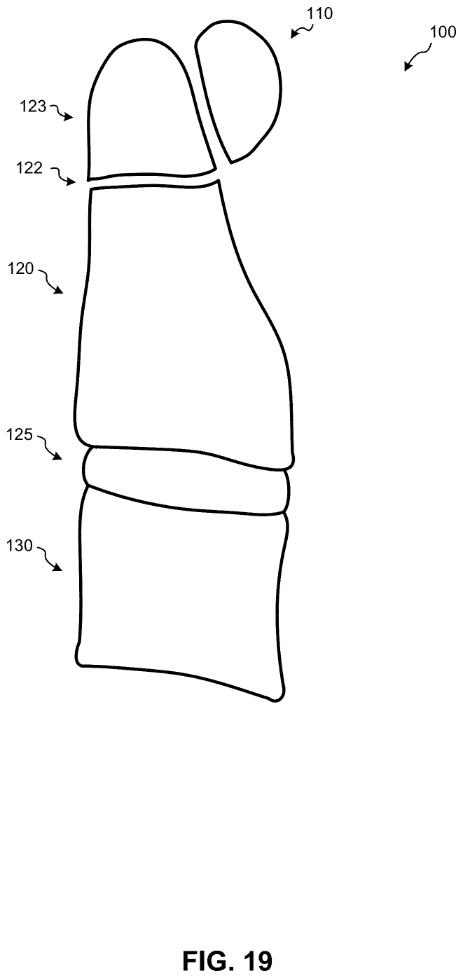

The odontoid guide cannula 210 may have a proximal end 211, a distal end 212, and a first longitudinal passage 214 extending through the odontoid guide cannula 210 between the proximal and distal ends 211, 212 of the odontoid guide cannula 210. The proximal end 211 of the odontoid guide cannula 210 may be coupled to the vertebra manipulator member 230, which may act as a handle. The vertebra manipulator member 230 may be configured to receive one or more first manipulation forces (not shown) from a surgeon to align a first vertebra (e.g., C3 130) of the patient relative to a second vertebra (e.g. C2 120) of the patient, as will be discussed below in more detail with reference to FIGS. 19-31. The vertebra manipulator member 230 may also be configured to receive one or more second manipulation forces (not shown) to align the second vertebra of the patient (e.g. C2 120) relative to a fractured odontoid process 123 located proximate the second vertebra 120. The distal end 212 of the odontoid guide cannula 210 may include a vertebra facing surface 216. In at lease on embodiment, the vertebra facing surface 216 may have an oval shape, as shown in FIG. 216. However, it will be understood that the vertebra facing surface 216 can have any other suitable shape. The vertebra facing surface 216 may be oriented relative to a longitudinal axis 201 of the odontoid guide cannula 210 at a first angle 241, as shown in FIG. 6H. The first angle 241 may be non-perpendicular to the longitudinal axis 201 and may range from between about zero degrees to about 90 degrees. In the particular embodiment shown in FIG. 6H, the first angle 241 may be somewhat greater than, or equal to, about 45 degrees.

The vertebra coupler 220 may be located at the distal end 212 of the odontoid guide cannula 210. The vertebra coupler 220 may be configured to couple with a first vertebra of a patient, such as C3 130 shown in FIGS. 1-3 and 19-31. In at least one embodiment, the vertebra coupler 220 may include a first spike 221 coupled to the vertebra facing surface 216. The first spike 221 may be oriented relative to the longitudinal axis 201 of the odontoid guide cannula 210 at a second angle 242, as shown in FIG. 6H. The vertebra coupler 220 may also include a second spike 222 coupled to the vertebra facing surface 216, opposite the first spike 221. The second spike 222 may also be oriented relative to the longitudinal axis 201 of the odontoid guide cannula 210 at the second angle 242. The second angle 242 may be non-perpendicular to the longitudinal axis 201, and may range from between about zero degrees to about 90 degrees. In the particular embodiment shown in FIG. 6H, the second angle 242 may be less than, or equal to, about 45 degrees. The vertebra coupler 220 may also include one or more intra-florescent markers (not shown) to help guide a surgeon in placing the vertebra coupler 220 proximate the first vertebra during a surgical procedure, via any suitable fluoroscopic imaging technique known in the art.

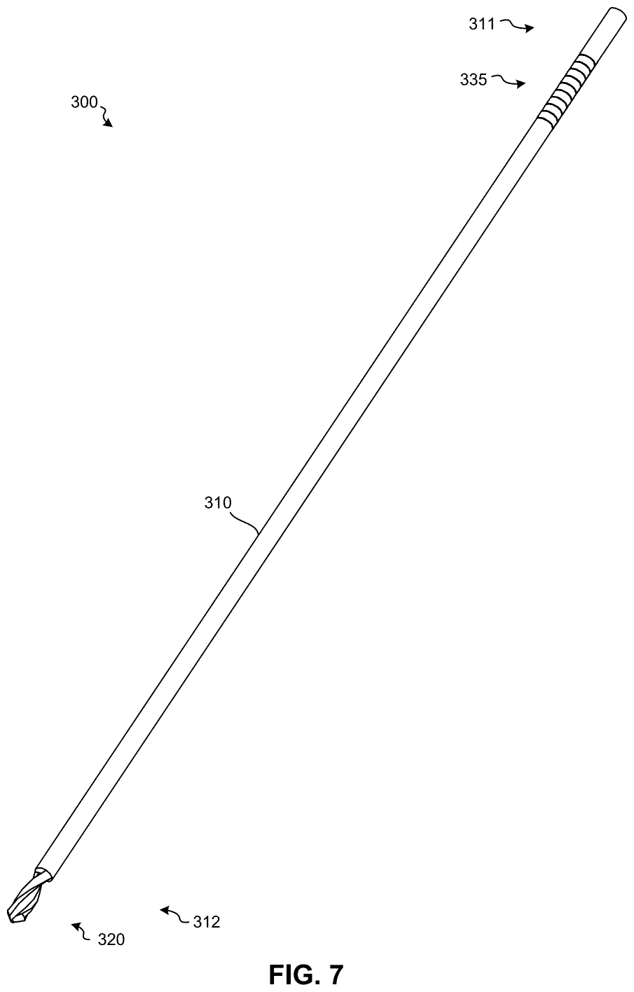

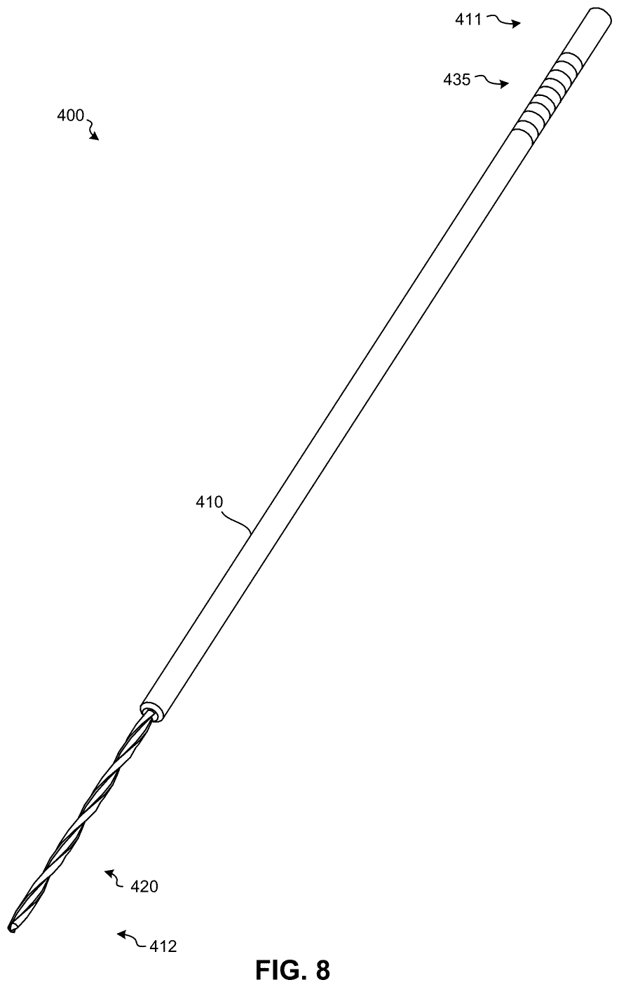

FIGS. 7 and 8 illustrate two drill bits 300, 400 that may be utilized with the odontoid fracture repair apparatus and systems of the present disclosure. Specifically, FIG. 7 illustrates a perspective view of a drill bit 300 and FIG. 8 illustrates a perspective view of a drill bit 400. The drill bit 300 may generally include a first shaft 310 having a proximal end 311, a distal end 312, a scale 335 located at the proximal end 311 of the first shaft 310, and a first drill bit head 320 coupled to the distal end 312 of the first shaft 310. Likewise, the drill bit 400 may generally include a second shaft 410 having a proximal end 411, a distal end 412, a scale 435 located at the proximal end 411 of the second shaft 410, and a second drill bit head 420 coupled to the distal end 412 of the second shaft 410. The first drill bit head 320 may have a greater diameter than the second drill bit head 420. In a particular embodiment, the first drill bit head 320 may have a diameter of about 6 mm and the second drill bit head 420 may have a diameter of about 3 mm. However, it will be understood that any diameter may be utilized for the first and second drill bit heads 320, 420. The use of drill bits 300, 400 will be described in more detail below with respect to FIGS. 19-31.

The scales 335, 435 located at the proximal ends 311, 411 of the first and second shafts 310, 410 of the first and second drill bits 300, 400 may be utilized by a surgeon to control the insertion depths of the drill bits 300, 400 through the odontoid guide 200, and into the patient, in order to prevent accidental damage to the patient's spine via over-insertion of the drill bits 300, 400 into the patient's spine. Moreover, each tool described herein for insertion through the odontoid guide 200 can include a similar scale to help the surgeon prevent over-insertion of the tool through the odontoid guide 200 and into the patient's spine. The scales associated with any tool described herein may comprise a series of markers spaced apart from each other at regular intervals in order to indicate a plurality of different insertion depths. The markers may be spaced apart from each other according to any desired scheme or distance. In one non-limiting example, the markers may be spaced apart from each other at regular intervals of about 5 mm. However, it will be understood that any distance between adjacent markers may be utilized, as desired.

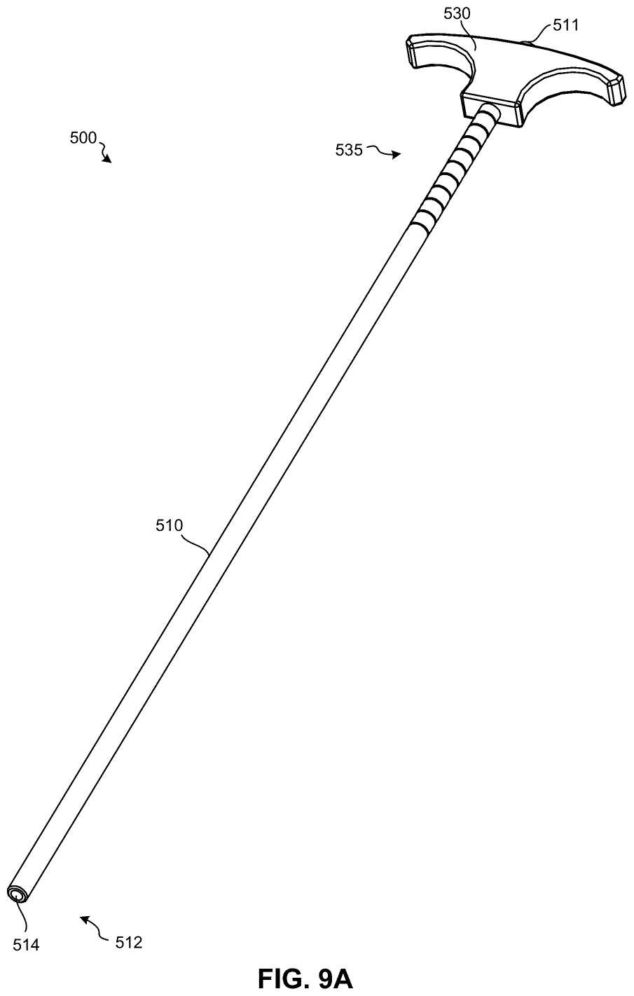

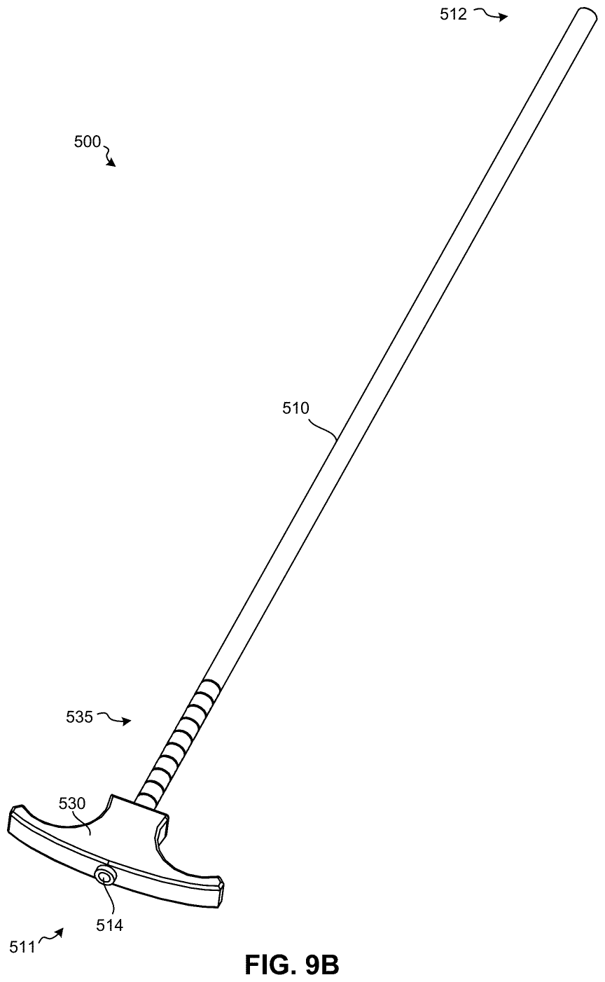

FIGS. 9A-9B illustrate various views of a bone cement retainer 500, according to an embodiment of the disclosure. Specifically, FIG. 9A illustrates a perspective view of a distal end of the bone cement retainer 500, and FIG. 9B illustrates a perspective view of a proximal end of the bone cement retainer 500. The bone cement retainer 500 may generally include a bone cement cannula 510 and a bone cement retainer handle 530.

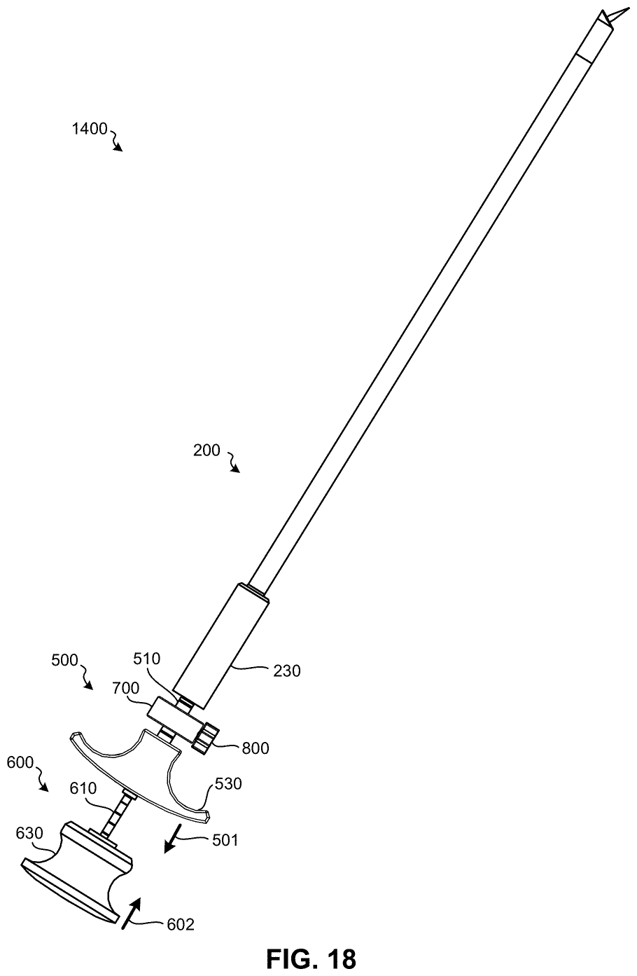

The bone cement cannula 510 may have a proximal end 511, a distal end 512, a scale 535, and a second longitudinal passage 514. The second longitudinal passage 514 may extend through the bone cement cannula 510 between the proximal and distal ends 511, 512 of the bone cement cannula 510. The second longitudinal passage 514 may be configured to receive bone cement (not shown) within the second longitudinal passage 514. The bone cement retainer handle 530 may also be coupled to the proximal end 511 of the bone cement cannula 510. The bone cement retainer handle 530 may be configured to receive a first force directed proximally, as will be discussed in more detail with respect to FIG. 18.

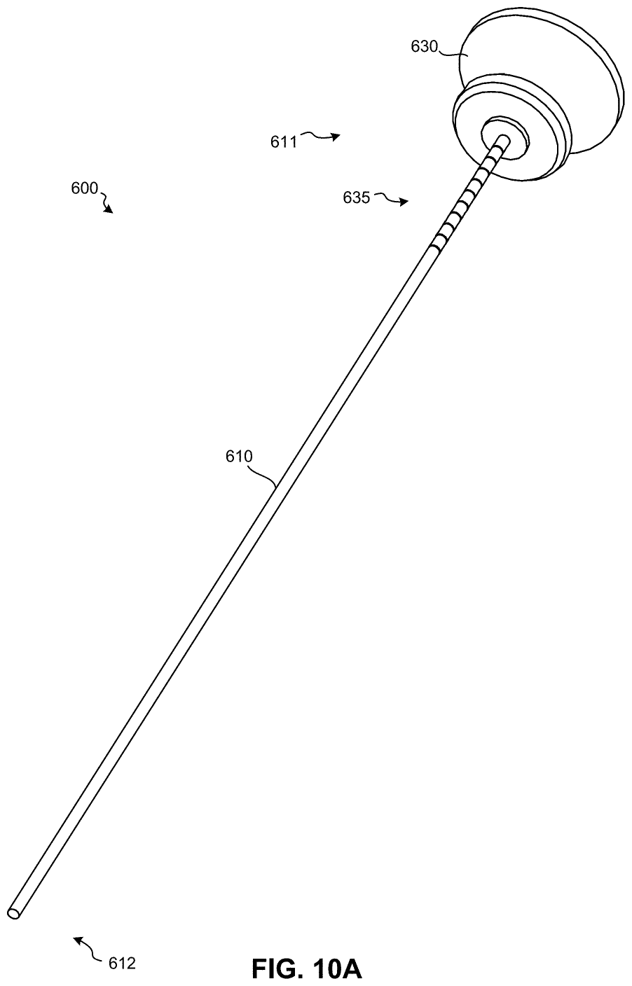

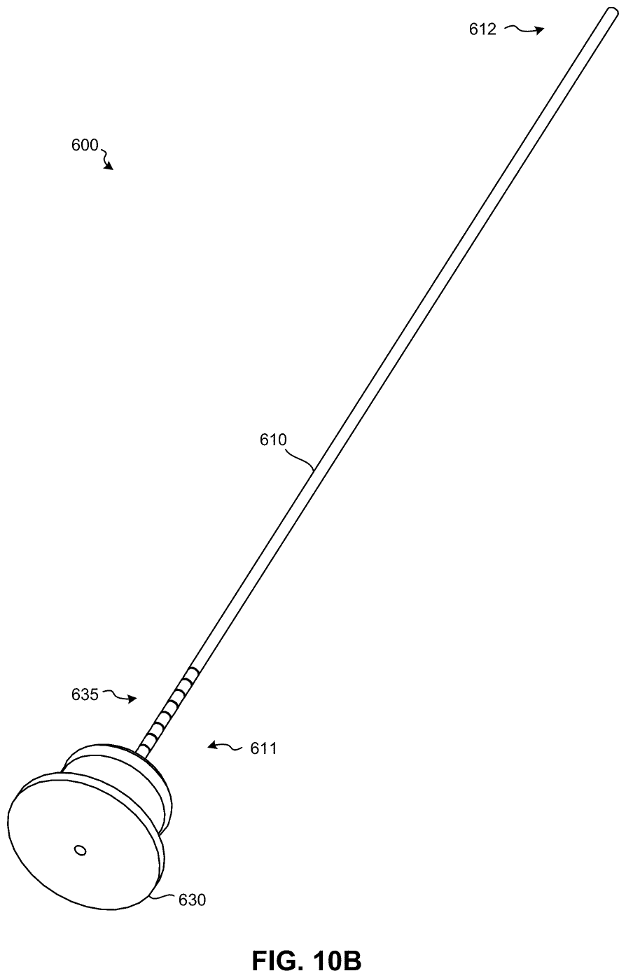

FIGS. 10A-10B illustrate various views of a bone cement ejector 600, according to an embodiment of the disclosure. Specifically, FIG. 10A is a perspective view of a distal end of the bone cement ejector 600, and FIG. 10B is a perspective view of a proximal end of the bone cement ejector 600. The bone cement ejector 600 may generally include a bone cement plunger 610 and a bone cement plunger handle 630.

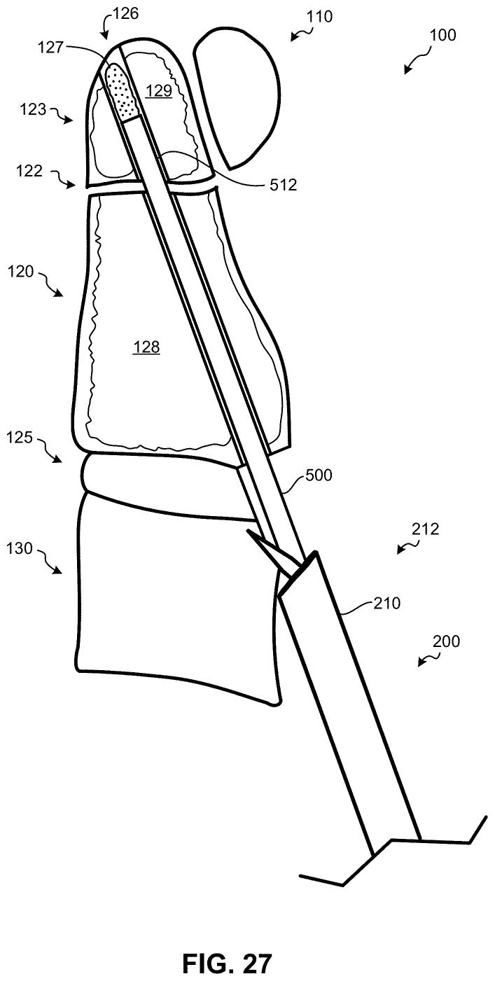

The bone cement plunger 610 may have a proximal end 611, a distal end 612, and a scale 635. In at least one embodiment, the bone cement plunger 610 may have a solid construction, with no longitudinal passage extending through the bone cement plunger 610. The bone cement plunger handle 630 may be coupled to the proximal end 611 of the bone cement plunger 610. The bone cement plunger handle 630 may be configured to receive a second force directed distally, in opposition to the first force. The second force may move the bone cement plunger 610 distally with respect to the bone cement cannula 510 in order to eject bone cement from the distal end 512 of the bone cement cannula 510, as will be discussed in more detail with respect to FIGS. 18 and 27.

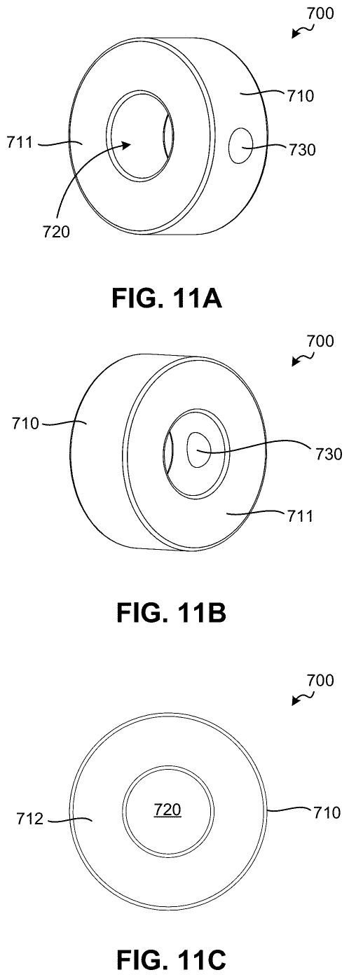

FIGS. 11A-11C illustrate various views of an adjustable stop member 700, according to an embodiment of the disclosure. Specifically, FIG. 11A is a perspective right side view of the adjustable stop member 700; FIG. 11B is a perspective left side view of the adjustable stop member 700; and FIG. 11C is a distal end view of the adjustable stop member 700. The adjustable stop member 700 may generally include a proximal surface 711, a distal surface 712, and a peripheral surface 710 extending from the proximal surface 711 to the distal surface 712.

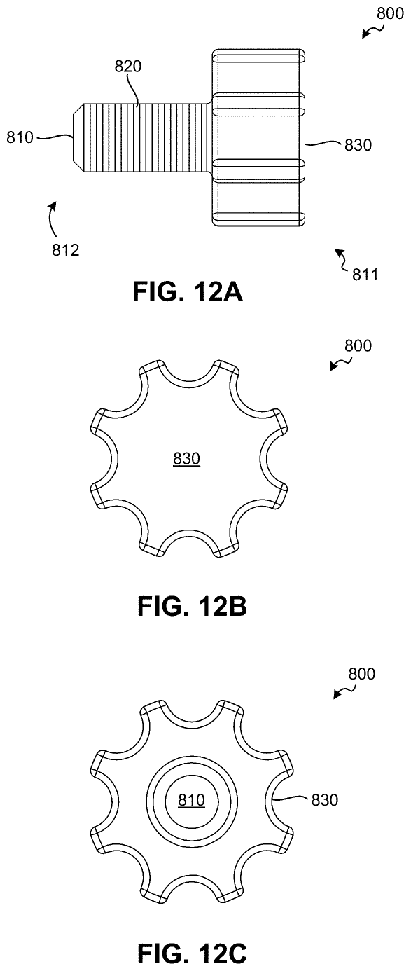

The adjustable stop member 700 may also include a first aperture 720 formed through the adjustable stop member 700 between the proximal surface 711 and the distal surface 712, and a second aperture 730 formed between the peripheral surface 710 and the first aperture 720, as can be seen in FIGS. 11A and 11B. The second aperture 730 may also include internal threading (not shown). The first aperture 720 may be configured to receive parts of the odontoid fracture repair apparatus and systems that are disclosed herein. For example, the first aperture 720 may receive at least the drill bit 300, the drill bit 400, the bone cement retainer 500, the bone cement ejector 600, and the bone screwdriver 1100. The second aperture 730 may be configured to receive a suitable stop member coupler, as will now be described below with reference to FIGS. 12A-12C.

FIGS. 12A-12C illustrate various views of a stop member coupler 800, according to an embodiment of the disclosure. Specifically, FIG. 12A is a side view of the stop member coupler 800; FIG. 12B is a proximal end view of the stop member coupler 800; and FIG. 12C is a distal end view of the stop member coupler 800. The stop member coupler 800 may generally include a coupler shaft 810 and a knob 830.

The coupler shaft 810 may have a proximal end 811, a distal end 812, and threading 820 formed about the coupler shaft 810 toward the distal end 812 of the coupler shaft 810. The knob 830 may be coupled to the proximal end 811 of the coupler shaft 810, and may be configured to receive one or more torque forces (not shown) from a surgeon to rotate the stop member coupler 800.

The threaded coupler shaft 810 may be inserted into the threaded second aperture 730 formed in the adjustable stop member 700 to removably engage the stop member coupler 800 with the adjustable stop member 700. Moreover, the coupler shaft 810 may be advanced into the second aperture 730 via rotation of the knob 830, until at least a portion of the distal end 812 of the coupler shaft 810 projects from the second aperture 730 and into the first aperture 720 of the adjustable stop member 700. Thus, the distal end 812 of the coupler shaft 810 may engage a tool of the odontoid fracture repair apparatus, that has been inserted through the first aperture 720 of the adjustable stop member 700, to couple the adjustable stop member 700 to the tool. As previously mentioned, the first aperture 720 may receive therethrough at least the drill bit 300, the drill bit 400, the bone cement retainer 500, the bone cement ejector 600, and the bone screwdriver 1100. In this manner, the adjustable stop member 700 may be selectively coupled to these tools at any point along each tool in order to limit how far each tool may project from the distal end 212 of the odontoid guide 200 (or project within the odontoid guide 200). In other words, once the adjustable stop member 700 has been coupled to a tool at a selected point along the tool, and the tool has been inserted into the odontoid guide 200, the distal surface 712 of the adjustable stop member 700 may engage the proximal end 211 of the odontoid guide cannula 210 (and/or engage the proximal end of the vertebra manipulator member 230) in order to prevent the tool from any further distal translation with respect to the odontoid guide 200.

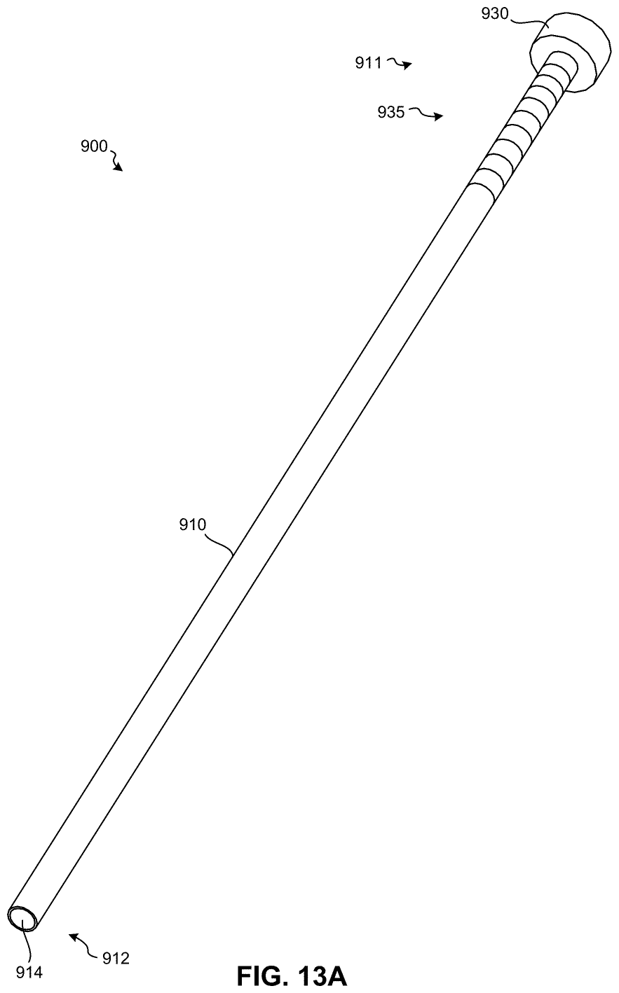

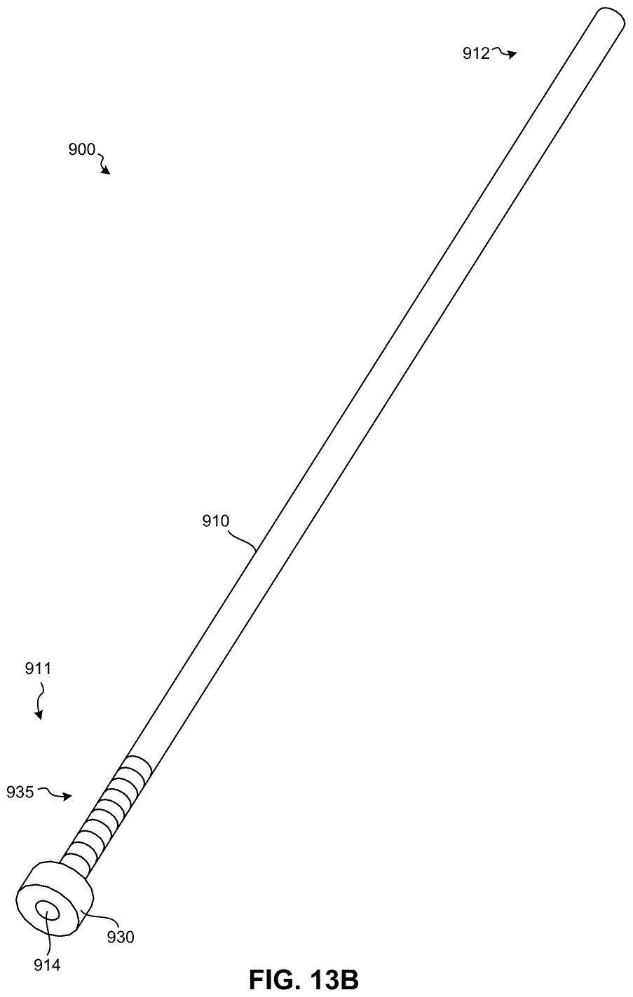

FIGS. 13A and 13B illustrate various views of an adapter sleeve 900, according to an embodiment of the disclosure. Specifically, FIG. 13A is a perspective view of a distal end of the adapter sleeve 900, and FIG. 13B is a perspective view of a proximal end of the adapter sleeve 900. In general, the adapter sleeve 900 may include an adapter sleeve cannula 910 and a stop

The adapter sleeve cannula 910 may have a proximal end 911, a distal end 912, a scale 935, and a third longitudinal passage 914 extending through the adapter sleeve cannula 910 between the proximal and distal ends 911, 912 of the adapter sleeve cannula 910. In at least one embodiment, the first longitudinal passage 214 of the odontoid guide 200 may be configured to receive the adapter sleeve cannula 910 therein. Moreover, the stop member 930 may be coupled to the proximal end 911 of the adapter sleeve cannula 910 in order to prevent the adapter sleeve cannula 910 from excessive distal translation with respect to the odontoid guide 200, in a similar manner to that discussed above with respect to the adjustable stop member 700.

The third longitudinal passage 914 of the adapter sleeve cannula 910 may be configured to receive a bone cement delivery system therein, such as the bone cement retainer 500 and/or the bone cement ejector 600 shown in FIGS. 9A-10B. In this manner, the adapter sleeve cannula 910 may allow for different bone cement delivery systems of varying size to properly fit within the first longitudinal passage 214 of the odontoid guide 200.

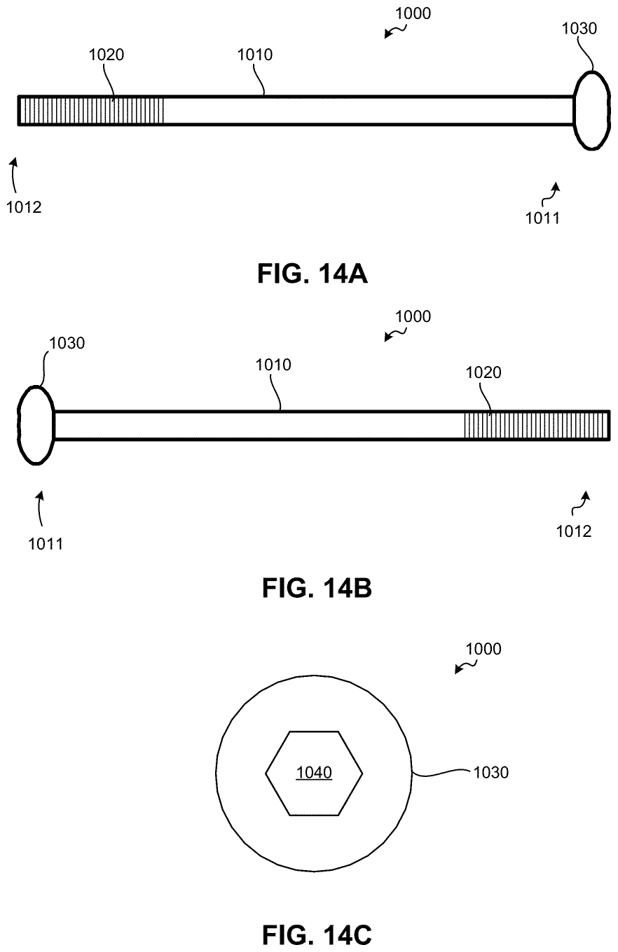

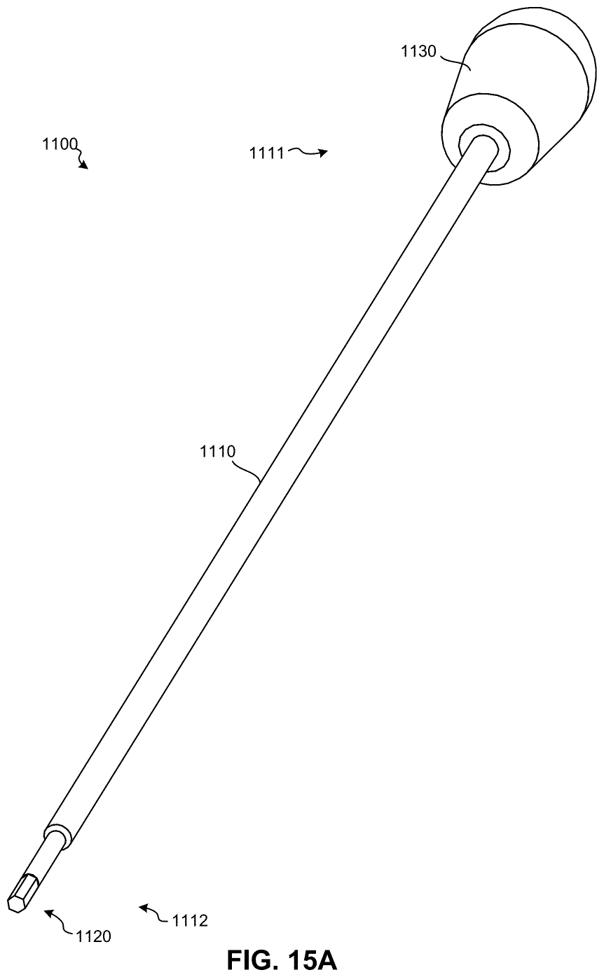

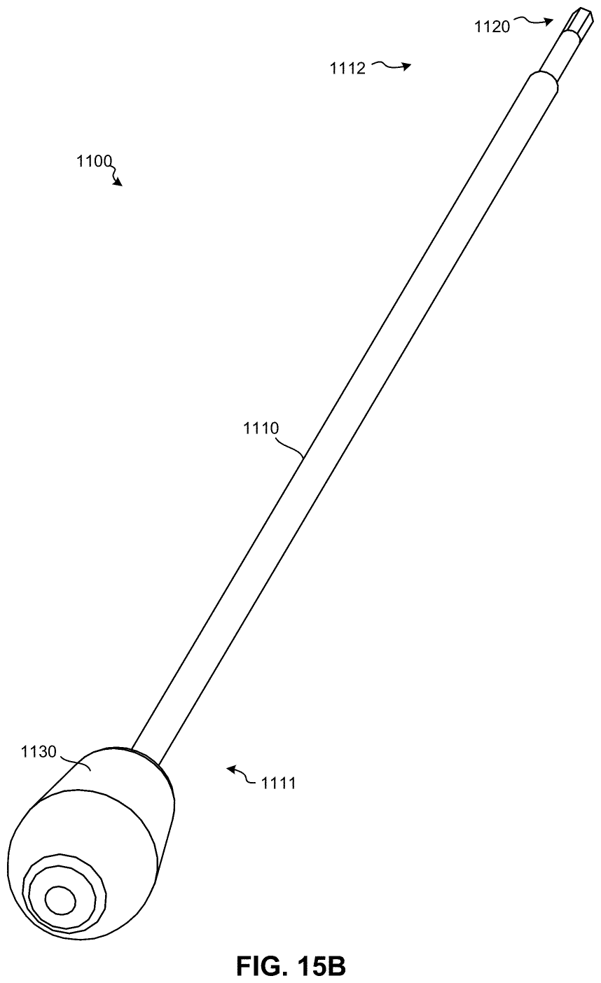

FIGS. 14A-14C illustrate various views of a bone screw 1000, according to an embodiment of the disclosure. Specifically, FIG. 14A is a right side view of the bone screw 1000; FIG. 14B is a left side view of the bone screw 1000; and FIG. 14C is a proximal end view of the bone screw 1000. In general, the bone screw 1000 may include a bone screw shaft 1010 and a bone screw head 1030. The bone screw shaft 1010 may have a proximal end 1011, a distal end 1012, and threading 1020 formed about the bone screw shaft 1010 toward the distal end 1012 of the bone screw shaft 1010. In other embodiments (not shown), the bone screw shaft 1010 may include threading 1020 formed about the bone screw shaft 1010 from the proximal end 1011 of the bone screw shaft 1010 to the distal end 1012 of the bone screw shaft 1010. The bone screw head 1030 may have a driver engagement feature 1040 formed therein that may be configured to receive a bone screw engagement feature of a suitable driver, as will now be discussed with respect to FIGS. 15A and 15B.

FIGS. 15A and 15B illustrate various views of a bone screwdriver 1100, according to an embodiment of the disclosure. Specifically, FIG. 15A is a perspective view of a distal end of the bone screwdriver 1100, and FIG. 15B is a perspective view of a proximal end of the bone screwdriver 1100. In general, the bone screwdriver 1100 may include a driver shaft 1110, a bone screw engagement feature 1120, and a driver handle 1130.

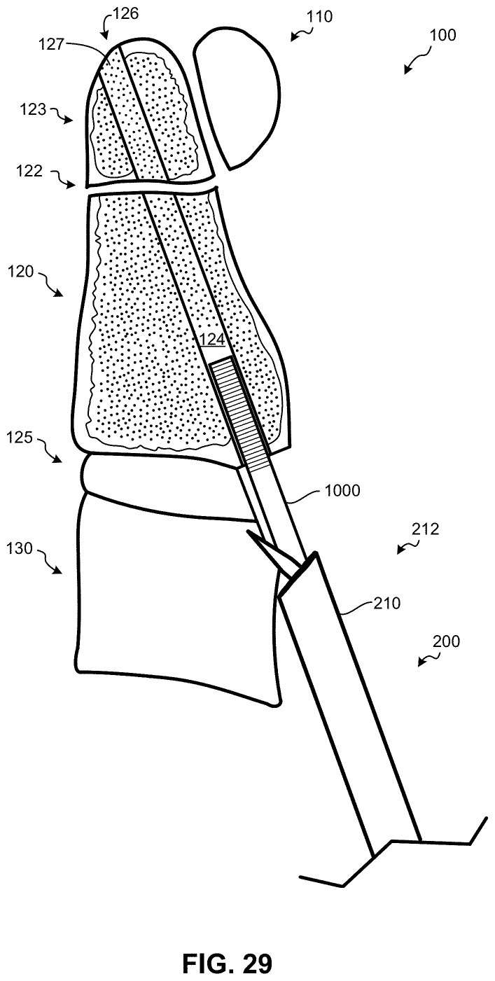

The driver shaft 1110 may include a proximal end 1111 and a distal end 1112. The bone screw engagement feature 1120 may be coupled to the distal end 1112 of the driver shaft 1110 and the driver handle 1130 may be coupled to the proximal end 1111 of the driver shaft 1110. While the bone screw engagement feature 1120 of the bone screwdriver 1100 and the driver engagement feature 1040 of the bone screw 1000 each have complementary hexagonal shapes, it will be understood that the bone screw engagement feature 1120 and the driver engagement feature 1040 may utilize any suitable complementary shape that is known in the art. The interaction between the bone screwdriver 1100 and the bone screw 1000 will be discussed in more detail with respect to FIGS. 29-31.

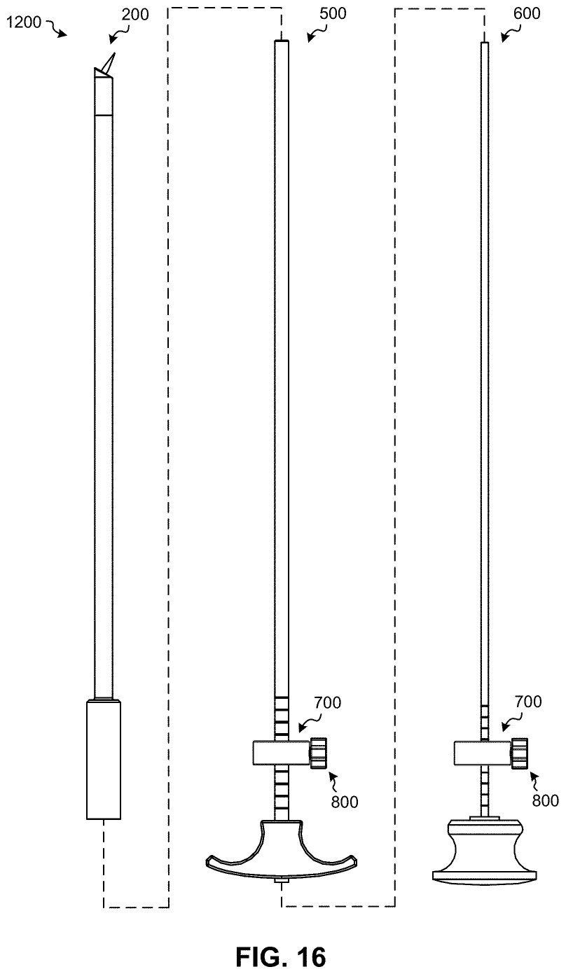

FIG. 16 illustrates an exploded view of an odontoid fracture repair system 1200, according to an embodiment of the disclosure. In this embodiment, the bone cement retainer 500 and the bone cement ejector 600 may together comprise a bone cement delivery system which may be directly receivable within the odontoid guide 200, as shown in the exploded view of FIG. 16. An adjustable stop member 700 and stop member coupler 800 are also shown coupled to the bone cement retainer 500 and the bone cement ejector 600 in FIG. 16, to illustrate how the adjustable stop member 700 and the stop member coupler 800 may be utilized to control the insertion depths of the bone cement retainer 500 and the bone cement ejector 600 through the odontoid guide 200.

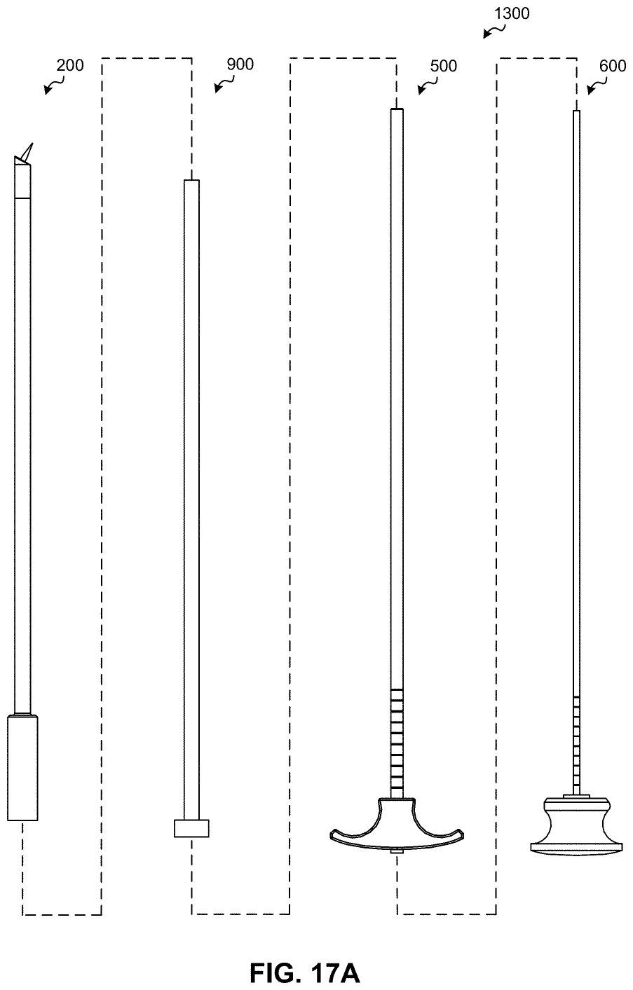

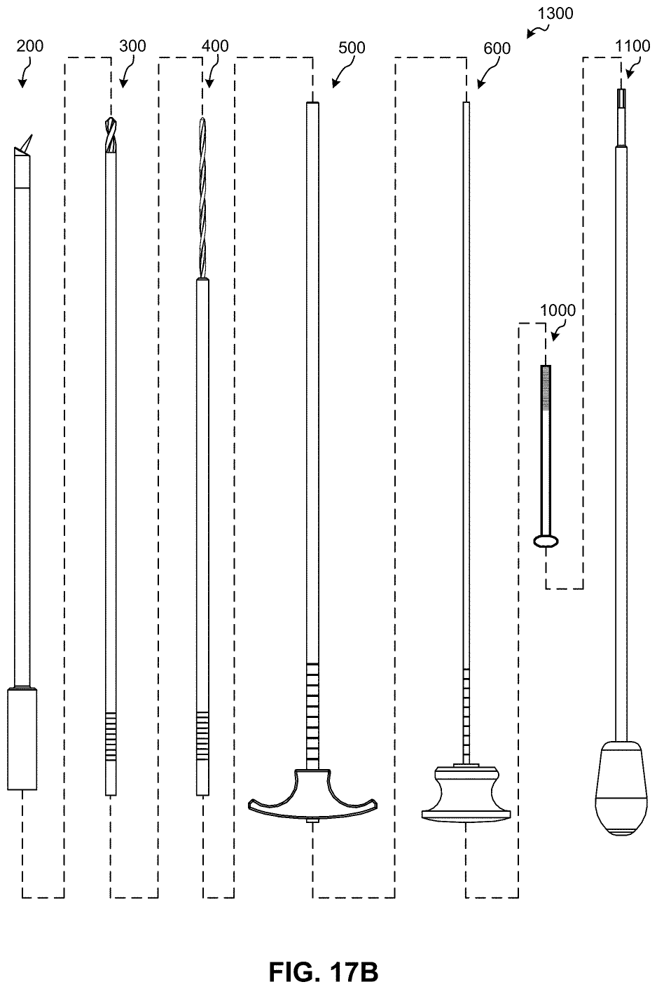

FIG. 17A illustrates an exploded view of an odontoid fracture repair system 1300, according to another embodiment of the disclosure. In this embodiment, the odontoid guide 200 and the adapter sleeve 900 may together comprise an odontoid guide fixation system, where the adapter sleeve 900 may be received within the odontoid guide 200. Moreover, the bone cement retainer 500 and the bone cement ejector 600 may together comprise a bone cement delivery system that may be receivable within the adapter sleeve 900. In this manner, the adapter sleeve 900 may allow for different sized bone cement delivery systems to properly fit within the odontoid guide 200, as previously discussed.

FIG. 17B is an exploded view of the odontoid fracture repair system 1300 of FIG. 17A including the drill bit 300, the drill bit 400, the bone screw 1000, and the bone screwdriver 1100 that may also be utilized with the odontoid fracture repair system 1300 shown in FIG. 17A. Thus, FIG. 17B illustrates an example insertion order for each part that may be associated with the odontoid fracture repair system 1300 during an example surgical procedure, as will be explained in more detail below with respect to FIGS. 19-31.

The all-in-one odontoid fracture repair system 1200, 1300 shown in FIGS. 16-17B illustrates another important advantage of this all in one odontoid fracture repair system 1200, 1300 over other known odontoid fracture repair systems. Specifically, this example all-in-one odontoid fracture repair system 1200, 1300 utilizes a unique set of components and fewer steps in comparison to other known odontoid fracture repair systems. For example, the all-in-one odontoid fracture repair system 1200 of FIG. 16 may not require any additional adapters in order to receive any of the tools for insertion through the odontoid guide 200. This will directly ligate the inherent risk to vital vascular and neuronal structures of the cervical spine, save valuable time of “under anesthesia time patient time,” and minimize retraction inherent iatrogenic trauma/edema to fragile surrounding structures of the neck during surgical procedures because the surgeon will not need to constantly insert and remove additional adapter sleeves to and from the odontoid guide 200 between each step of the surgical procedure. This may be accomplished by ensuring that the outer diameters of each tool for insertion through the odontoid guide 200 matches the inner diameter of the longitudinal passage of the odontoid guide 200. Thus, the odontoid guide fixation systems disclosed herein may not require any additional adapter sleeves in order to receive a bone cement delivery system within the longitudinal passage of the odontoid guide 200. Likewise, the odontoid fracture repair system 1300 of FIG. 17A may only utilize one adapter sleeve 900 in order to receive all of the tools for insertion through the odontoid guide 200, as shown in FIG. 17B. This will likewise save valuable time during surgical procedures because the surgeon will not need to constantly insert and remove any additional adapter sleeves to and from the odontoid guide 200 between each step of the surgical procedure. This may be accomplished by ensuring that the outer diameters of each tool for insertion through the odontoid guide 200 matches the inner diameter of the longitudinal passage of the adapter sleeve 900. Moreover, placement of the odontoid guides 200 within a patient for each of these odontoid fracture repair systems described herein may be accomplished without the use of any complex retractor systems in contrast to other known odontoid fracture repair systems, thus providing another novelty layer of safety, as well as saving additional time during surgical procedures (directly related to likelihood of successful patient extubation and minimizing the likelihood for dysphagia, and the need of post-operative placement of percutaneous endoscopic gastrostomy (PEG)/tracheostomy) that utilize the odontoid fracture repair systems disclosed herein.

FIG. 18 illustrates a perspective view of an assembled odontoid fracture repair system 1400, according to an embodiment of the disclosure. In general, the odontoid fracture repair system 1400 may include at least an odontoid guide 200, a bone cement retainer 500, and a bone cement ejector 600. The odontoid fracture repair system 1400 may also include one or more adjustable stop members 700 and stop member couplers 800 coupled to the bone cement retainer 500 and/or the bone cement ejector 600, as shown in FIG. 18. In certain embodiments, the odontoid fracture repair system 1400 may also include an adapter sleeve 900 (not shown in FIG. 18). The bone cement retainer 500 may be filled with bone cement (not shown) and the bone cement retainer 500 may then be inserted into the odontoid guide 200. The bone cement ejector 600 may then be inserted into the bone cement retainer 500.

As shown in FIG. 18, the bone cement retainer handle 530 may be configured to receive a first force 501 directed toward the proximal direction. The bone cement plunger handle 630 may also be configured to receive a second force 602 directed toward the distal direction, opposite the first force 501. In this manner, the bone cement plunger 610 may be moved distally inside of the bone cement cannula 510 in order to eject bone cement out of the distal end 512 of the bone cement cannula 510, as will be discussed in more detail with respect to FIG. 27.

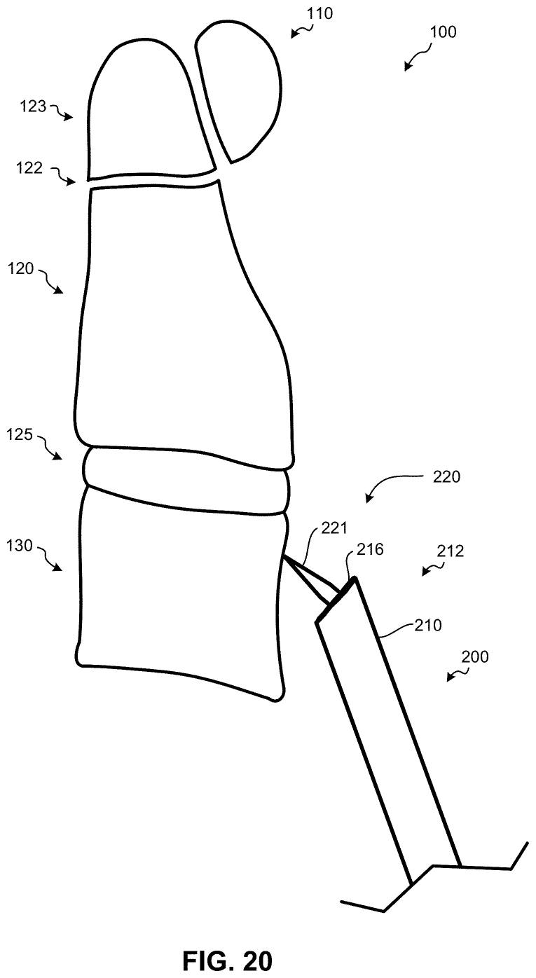

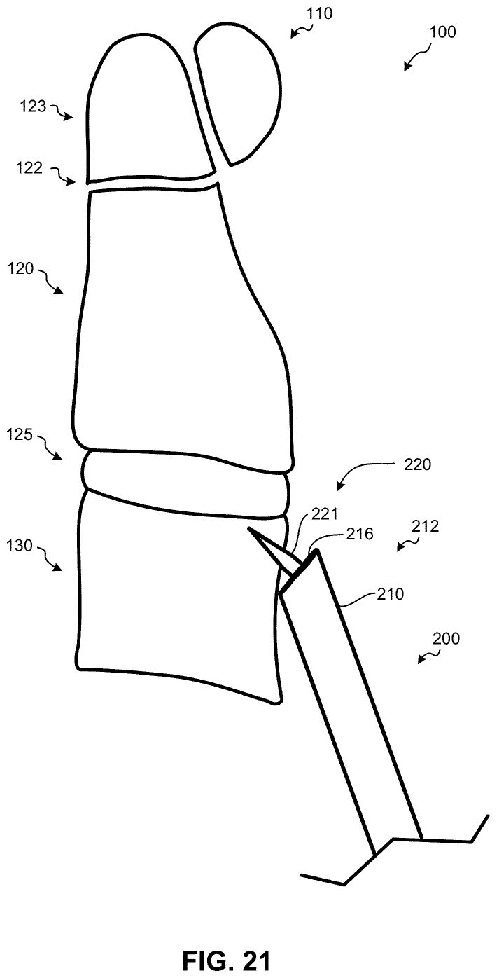

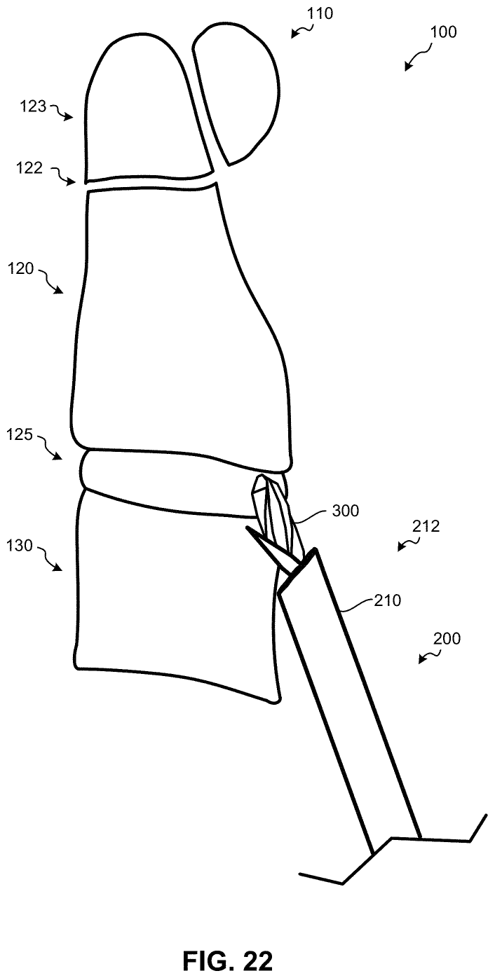

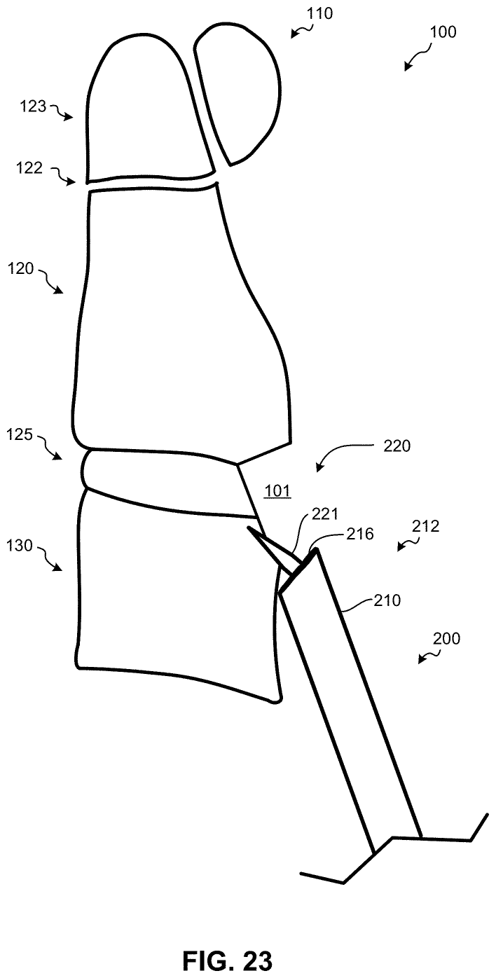

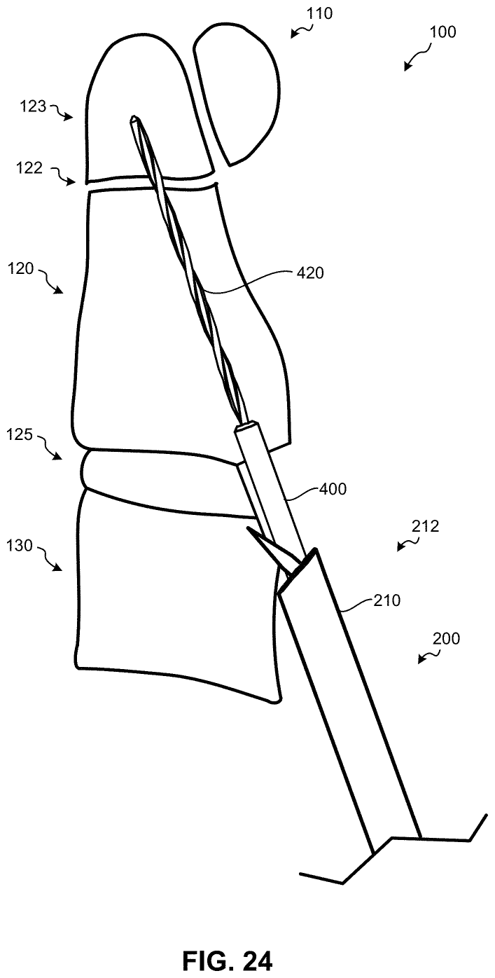

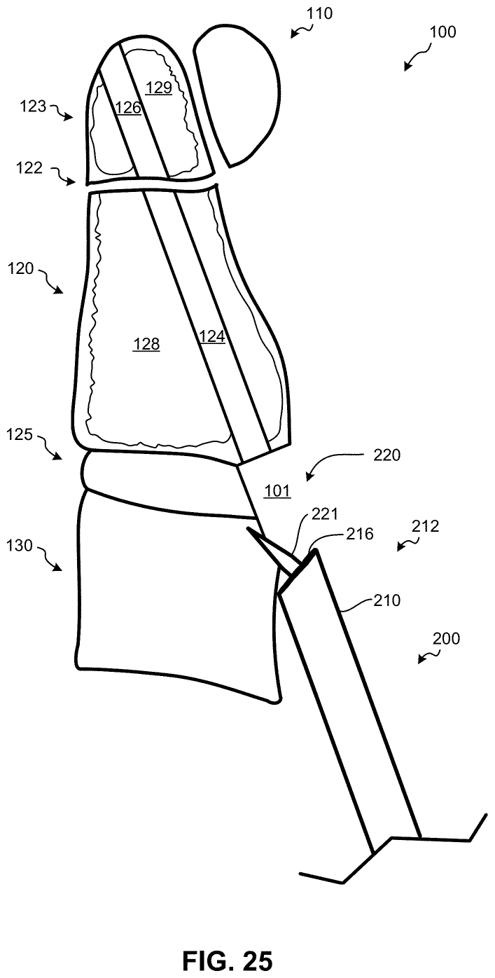

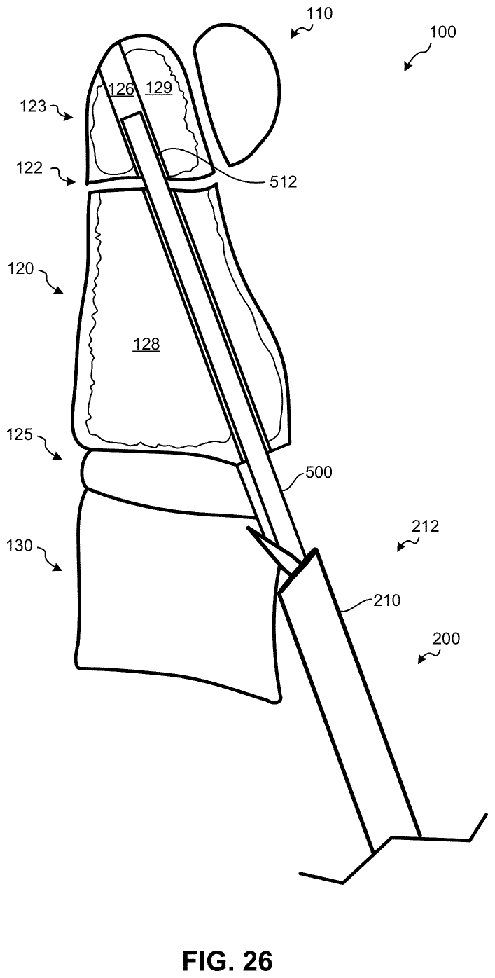

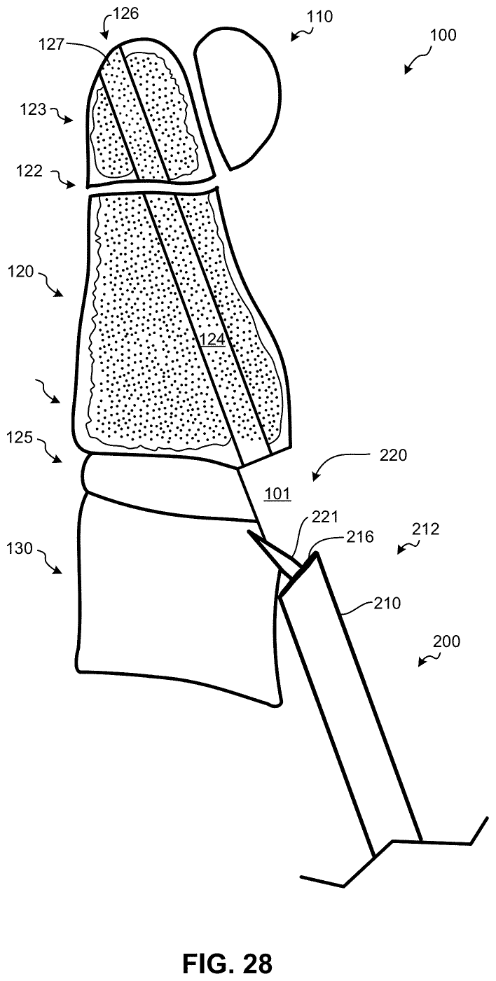

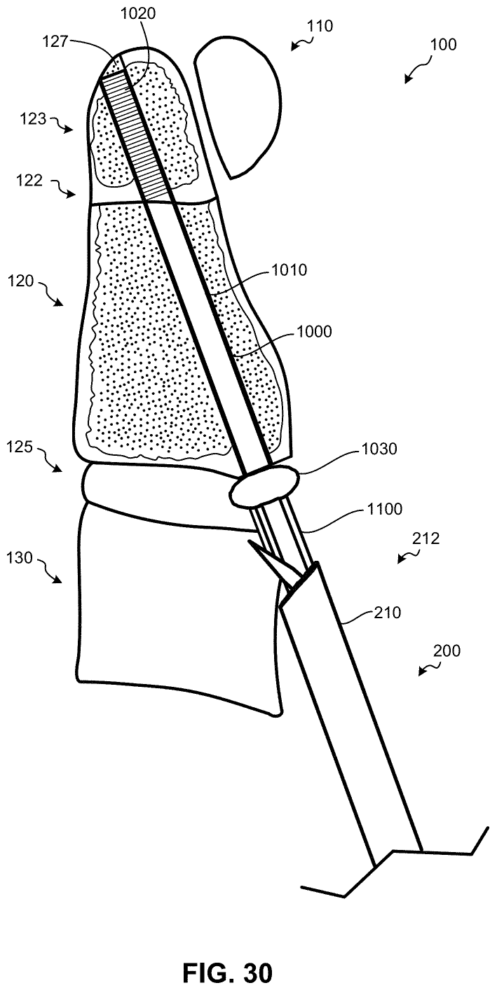

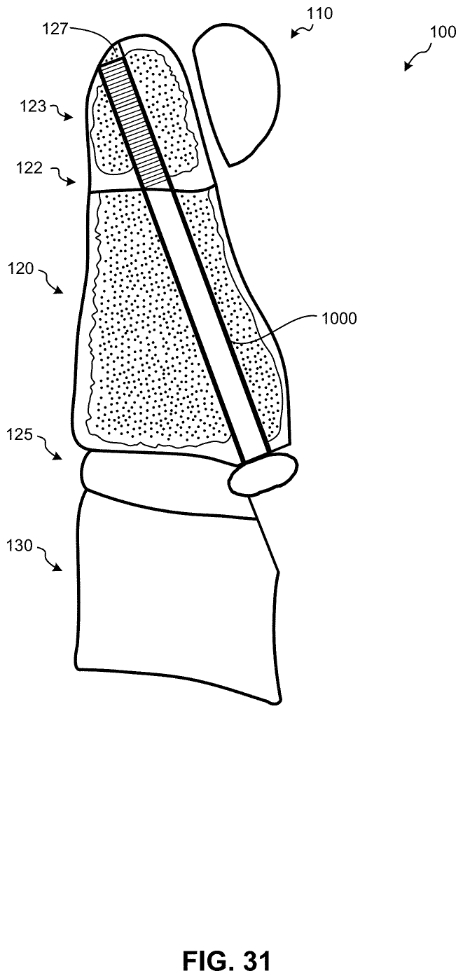

FIGS. 19-31 illustrate various steps for an example surgical procedure to repair an odontoid fracture 122 of a cervical spine 100, which may be performed from an anterior surgical approach. For example, FIG. 19 illustrates a portion of the cross sectional side view of the cervical spine 100 Shown in FIG. 1. In particular, FIG. 19 depicts a cross sectional side view of portions of the C1, C2, and C3 vertebrae of FIG. 1, taken along an anterior/posterior plane, including a type II odontoid process fracture 122 formed in the C2 vertebra 120. FIG. 20 shows the cervical spine 100 of FIG. 19 with a vertebra coupler 220 of an odontoid guide 200 placed proximate the C3 130 vertebra. FIG. 21 shows the vertebra coupler 220 after it has been driven into the C3 130 vertebra. This may be accomplished by: (1) placing the distal end 212 of the odontoid guide cannula 210 proximate the first vertebra C3 130 of the patient; and (2) coupling the odontoid guide to the first vertebra C3 130 by applying one or more impact forces (not shown) to the proximal end 211 of the odontoid guide 200 with a mallet (not shown) to drive the first and second spikes 221, 222 of the vertebra coupler 220 into C3 130 and couple the odontoid guide 200 to the first vertebra 3 130. FIG. 22 shows a drill bit 300 projecting from the distal end 212 of the odontoid guide 200 to remove portions of C2 120, C3 130, and the C2-C3 disc 125. FIG. 23 shows the drill bit 300 removed from the odontoid guide 200 to illustrate the portions of C2 120, C3 130, and the C2-C3 disc 125 that have been removed by the drill bit 300 in the space 101. FIG. 24 shows the drill bit 400 projecting from the distal end 212 of the odontoid guide 200 to form bone tunnels (see FIG. 25) through C2 120 and the fractured odontoid process 123, respectively. FIG. 25 shows the drill bit 400 removed from the odontoid guide 200 to illustrate the bone tunnels 124, 126 that have each been formed in C2 120 and the fractured odontoid process 123, respectively. FIG. 25 also shows porous intramedullary spaces 128, 129 that may be formed within C2 120 and the fractured odontoid process 123, respectively. FIG. 26 shows the distal end 512 of the bone cement retainer 500 projecting from the distal end 212 of the odontoid guide 200 and into the bone tunnels 124, 126. FIG. 27 shows bone cement 127 being ejected from the distal end 512 of the bone cement retainer 500 and into the bone tunnel 126. FIG. 28 shows the bone cement retainer 500 removed from the odontoid guide 200, with ejected bone cement 127 remaining inside the odontoid process 123 and/or C2. In one embodiment, the bone cement 127 may substantially reside within the bone tunnel 126. In another embodiment, the bone cement 127 may substantially reside within the bone tunnel 126 and the bone tunnel 124. In yet another embodiment, the bone cement 127 may reside within the bone tunnel 126 and within the porous intramedullary space 129 of the odontoid process 123. In still another embodiment, the bone cement 127 may reside within the bone tunnels 124, 126 and within the porous intramedullary spaces 128, 129 within C2 and the odontoid process 123, as is shown in FIG. 28. FIG. 29 shows a bone screw 1000 being inserted into the bone tunnels 124, 126 through the distal end 212 of the odontoid guide 200. FIG. 30 shows the bone screw 1000 fully inserted into the bone tunnels 124, 126 with the bone screwdriver 1100 securing the bone screw 1000 into place in order to reduce the odontoid fracture 122. FIG. 31 shows the bone screw 1000 fully seated within the bone tunnels 124, 126 and the odontoid fracture 122 fully reduced with the bone screwdriver 1100 and odontoid guide 200 removed from the patient. In this manner, the bone cement 127 can help secure the bone screw 1000 to the odontoid process 123 and improve the strength and resiliency of the odontoid fracture repair.

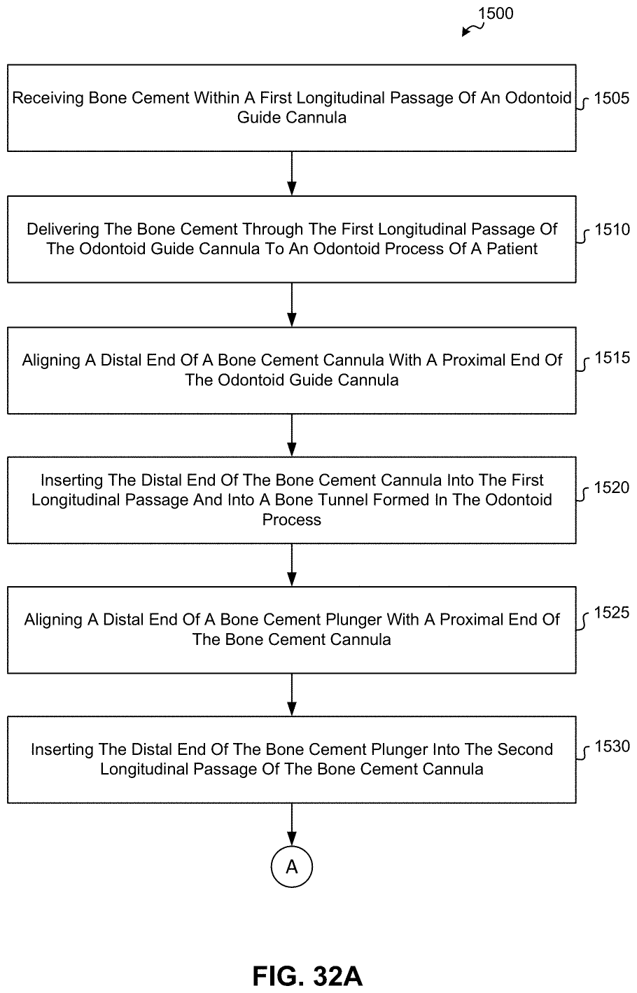

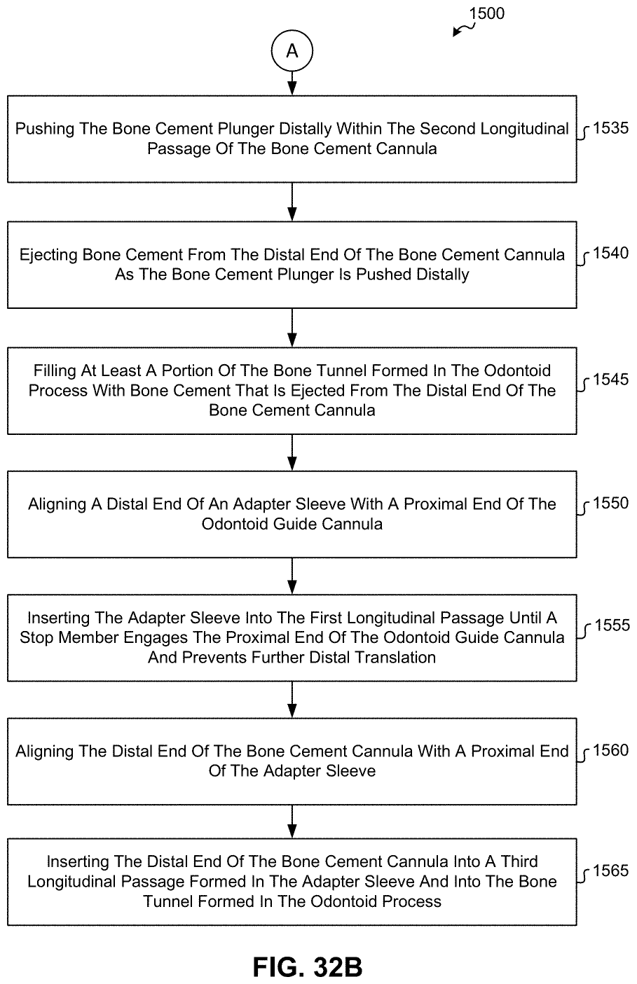

FIGS. 32A-32B illustrate a flowchart of a method 1500 for repairing an odontoid fracture in a patient, according to an embodiment of the disclosure. In general, the method 1500 may include the use of an odontoid guide that comprises an odontoid guide cannula having a first longitudinal passage.

The method 1500 may begin with a step 1505 in which bone cement may be received within the first longitudinal passage of the odontoid guide cannula. In an embodiment, the bone cement may be received directly by the first longitudinal passage of the odontoid guide cannula. However, in other embodiments the bone cement may be directly received by a second longitudinal passage of a bone cement cannula. The bone cement cannula, with bone cement inside the second longitudinal passage, may then be received within the first longitudinal passage of the odontoid guide cannula.

Once the bone cement has been received within the first longitudinal passage of the odontoid guide cannula, the method 1500 may proceed to a step 1510 in which the bone cement may be delivered through the first longitudinal passage of the odontoid guide cannula to an odontoid process of the patient. In an embodiment, the bone cement may be delivered directly through the first longitudinal passage of the odontoid guide cannula. However, in other embodiments, the bone cement may be delivered directly through the second longitudinal passage of the bone cement cannula, which has been received within the first longitudinal passage of the odontoid guide cannula.

Once the bone cement has been delivered through the first longitudinal passage of the odontoid guide cannula to an odontoid process of the patient, the method 1500 may end.

Alternatively, or in addition thereto, the method 1500 may further proceed to any or all of the remaining steps shown in FIGS. 32A and 32B. For example, the method 1500 may proceed to a step 1515 in which a distal end of the bone cement cannula may be aligned with a proximal end of the odontoid guide cannula.

Once the distal end of the bone cement cannula has been aligned with a proximal end of the odontoid guide cannula, the method 1500 may proceed to a step 1520 in which the distal end of the bone cement cannula may be inserted into the first longitudinal passage of the odontoid guide cannula until the distal end of the bone cement cannula is received within a bone tunnel formed in the odontoid process of the patient.

Once the distal end of the bone cement cannula has been inserted into the first longitudinal passage of the odontoid guide cannula until the distal end of the bone cement cannula has been received within the bone tunnel formed in the odontoid process of the patient, the method 1500 may proceed to a step 1525 in which a distal end of a bone cement plunger may be aligned with a proximal end of the bone cement cannula.

Once the distal end of the bone cement plunger has been aligned with the proximal end of the bone cement cannula, the method 1500 may proceed to a step 1530 in which the distal end of the bone cement plunger may be inserted into the second longitudinal passage of the bone cement cannula.

Once the distal end of the bone cement plunger has been inserted into the second longitudinal passage of the bone cement cannula, the method 1500 may proceed to a step 1535 in which the bone cement plunger may be pushed distally within the second longitudinal passage of the bone cement cannula.

Once the bone cement plunger has been pushed distally within the second longitudinal passage of the bone cement cannula, the method 1500 may proceed to a step 1540 in which bone cement may be ejected from the distal end of the bone cement cannula as the bone cement plunger is pushed distally.

Once the bone cement has been ejected from the distal end of the bone cement cannula as the bone cement plunger was pushed distally, the method 1500 may proceed to a step 1545 in which at least a portion of the bone tunnel formed in the odontoid process may be filled with bone cement that has been ejected from the distal end of the bone cement cannula, and the method 1500 may end.

Alternatively, or in addition thereto, the method 1500 may proceed to a step 1550 in which a distal end of an adapter sleeve may be aligned with a proximal end of the odontoid guide cannula.

Once the distal end of an adapter sleeve had been aligned with the proximal end of the odontoid guide cannula, the method 1500 may proceed to a step 1555 in which the adapter sleeve may be inserted into the first longitudinal passage of the odontoid guide cannula until a stop member of the adapter sleeve engages the proximal end of the odontoid guide cannula and prevents the adapter sleeve from further distal translation with respect to the odontoid guide.

Once the adapter sleeve has been fully inserted into the first longitudinal passage of the odontoid guide cannula, the method 1500 may proceed to a step 1560 in which the distal end of the bone cement cannula may be aligned with a proximal end of the adapter sleeve.

Once the distal end of the bone cement cannula has been aligned with the proximal end of the adapter sleeve, the method 1500 may proceed to a step 1565 in which the distal end of the bone cement cannula may be inserted into a third longitudinal passage formed in the adapter sleeve until the distal end of the bone cement cannula is received within the bone tunnel formed in the odontoid process of the patient, and the method 1500 may end.

Any methods disclosed herein comprise one or more steps or actions for performing the described method. One or more of the method steps and/or actions may be omitted from any of the methods disclosed herein. Moreover, any of the method steps and/or actions may be interchanged with one another. In other words, unless a specific order of steps or actions is required for proper operation of the embodiment, the order and/or use of specific steps and/or actions may be modified.

Reference throughout this specification to “an embodiment” or “the embodiment” means that a particular feature, structure or characteristic described in connection with that embodiment is included in at least one embodiment. Thus, the quoted phrases, or variations thereof, as recited throughout this specification are not necessarily all referring to the same embodiment.

Similarly, it should be appreciated that in the above description of embodiments, various features are sometimes grouped together in a single embodiment, Figure, or description thereof for the purpose of streamlining the disclosure. This method of disclosure, however, is not to be interpreted as reflecting an intention that any claim requires more features than those expressly recited in that claim. Rather, as the following claims reflect, inventive aspects lie in a combination of fewer than all features of any single foregoing disclosed embodiment. Thus, the claims following this Detailed Description are hereby expressly incorporated into this Detailed Description, with each claim standing on its own as a separate embodiment. This disclosure includes all permutations of the independent claims with their dependent claims.

Recitation in the claims of the term “first” with respect to a feature or element does not necessarily imply the existence of a second or additional such feature or element. Elements recited in means-plus-function format are intended to be construed in accordance with 35 U.S.C. § 112 Para. 6. It will be apparent to those having skill in the art that changes may be made to the details of the above-described embodiments without departing from the underlying principles set forth herein.

While specific embodiments and applications of the present disclosure have been illustrated and described, it is to be understood that the scope of the appended claims is not limited to the precise configuration and components disclosed herein. Various modifications, changes, and variations which will be apparent to those skilled in the art may be made in the arrangement, operation, and details of the apparatus, systems, and methods disclosed herein.

What is claimed is:

1. An all-in-one odontoid fracture repair system comprising:
an odontoid guide fixation system, the odontoid guide fixation system comprising:
an odontoid guide, the odontoid guide comprising:
an odontoid guide cannula having a proximal end and a distal end;
a first longitudinal passage extending through the odontoid guide cannula between the proximal and distal ends of the odontoid guide cannula; and
a vertebra coupler located at the distal end of the odontoid guide cannula, the vertebra coupler configured to couple with a first vertebra of a patient; and
a bone cement delivery system receivable within the first longitudinal passage of the odontoid guide cannula, the bone cement delivery system comprising:
a bone cement retainer, the bone cement retainer comprising:
a bone cement cannula having a proximal end and a distal end;
a second longitudinal passage extending through the bone cement cannula between the proximal and distal ends of the bone cement cannula, the second longitudinal passage configured to receive bone cement therein; and
a bone cement retainer handle coupled to the proximal end of the bone cement cannula, the bone cement retainer handle configured to receive a first force directed proximally; and
a bone cement ejector, the bone cement ejector comprising:
a bone cement plunger having a proximal end and a distal end, the bone cement plunger configured to be received within the second longitudinal passage of the bone cement cannula; and
a bone cement plunger handle coupled to the proximal end of the bone cement plunger, the bone cement plunger handle configured to receive a second force directed distally, in opposition to the first force, to move the bone cement plunger distally with respect to the bone cement cannula and eject the bone cement from the distal end of the bone cement cannula.

2. The all-in-one odontoid fracture repair system of claim 1, wherein the distal end of the odontoid guide cannula comprises a vertebra facing surface, wherein the vertebra facing surface is oriented non-perpendicular to a longitudinal axis of the odontoid guide cannula at a first angle.

3. The all-in-one odontoid fracture repair system of claim 2, wherein the vertebra coupler comprises:
a first spike coupled to the vertebra facing surface, the first spike oriented relative to the longitudinal axis of the odontoid guide cannula at a second angle; and
a second spike coupled to the vertebra facing surface, opposite the first spike, the second spike oriented relative to the longitudinal axis of the odontoid guide cannula at the second angle.

4. The all-in-one odontoid fracture repair system of claim 1, further comprising a vertebra manipulator member coupled to the proximal end of the odontoid guide cannula, the vertebra manipulator member configured to:
receive one or more first manipulation forces to align the first vertebra of the patient relative to a second vertebra of the patient; and
receive one or more second manipulation forces to align the second vertebra relative to a fractured odontoid process located proximate the second vertebra.

5. The all-in-one odontoid fracture repair system of claim 1, wherein the odontoid guide fixation system does not require any additional adapter sleeves to receive the bone cement delivery system within the first longitudinal passage of the odontoid guide cannula.

6. The all-in-one odontoid fracture repair system of claim 1, wherein the odontoid guide fixation system further comprises:
an adapter sleeve, the adapter sleeve comprising:
an adapter sleeve cannula having a proximal end and a distal end; and
a third longitudinal passage extending through the adapter sleeve cannula between the proximal and distal ends of the adapter sleeve cannula,
wherein the odontoid guide is configured to receive the adapter sleeve cannula within the first longitudinal passage of the odontoid guide cannula, and
wherein the adapter sleeve is configured to receive the bone cement delivery system within the third longitudinal passage of the adapter sleeve cannula.

7. The all-in-one odontoid fracture repair system of claim 6 wherein, the adapter sleeve comprises a stop member coupled to the proximal end of the adapter sleeve cannula, wherein the stop member is configured to engage the proximal end of the odontoid guide cannula and prevent the adapter sleeve from further distal translation with respect to the odontoid guide.

8. An odontoid fracture repair apparatus comprising:
an odontoid guide, the odontoid guide comprising:
an odontoid guide cannula having a proximal end and a distal end;
a first longitudinal passage extending through the odontoid guide cannula between the proximal and distal ends of the odontoid guide cannula; and
a vertebra coupler located at the distal end of the odontoid guide cannula, the vertebra coupler configured to couple to a first vertebra of a patient,
wherein the odontoid guide is configured to receive a bone cement delivery system within the first longitudinal passage of the odontoid guide cannula.

9. The odontoid fracture repair apparatus of claim 8, wherein the distal end of the odontoid guide cannula comprises a vertebra facing surface, wherein the vertebra facing surface is oriented non-perpendicular to a longitudinal axis of the odontoid guide cannula at a first angle.

10. The odontoid fracture repair apparatus of claim 9, wherein the vertebra coupler comprises:
a first spike coupled to the vertebra facing surface, the first spike oriented relative to the longitudinal axis of the odontoid guide cannula at a second angle; and
a second spike coupled to the vertebra facing surface, opposite the first spike, the second spike oriented relative to the longitudinal axis of the odontoid guide cannula at the second angle.

11. The odontoid fracture repair apparatus of claim 8, further comprising a vertebra manipulator member coupled to the proximal end of the odontoid guide cannula, the vertebra manipulator member configured to:
receive one or more first manipulation forces to align the first vertebra of the patient relative to a second vertebra of the patient; and
receive one or more second manipulation forces to align the second vertebra relative to a fractured odontoid process located proximate the second vertebra.

12. The odontoid fracture repair apparatus of claim 8, wherein the odontoid fracture repair apparatus does not require any additional adapter sleeves to receive a bone cement delivery system within the first longitudinal passage of the odontoid guide cannula.

13. The odontoid fracture repair apparatus of claim 8 further comprising:
an adapter sleeve, the adapter sleeve comprising:
an adapter sleeve cannula having a proximal end and a distal end; and
a second longitudinal passage extending through the adapter sleeve cannula between the proximal and distal ends of the adapter sleeve cannula,
wherein the odontoid guide is configured to receive the adapter sleeve cannula within the first longitudinal passage of the odontoid guide cannula, and
wherein the adapter sleeve is configured to receive the bone cement delivery system within the second longitudinal passage of the adapter sleeve cannula.

14. The odontoid fracture repair apparatus of claim 13 wherein, the adapter sleeve comprises a stop member coupled to the proximal end of the adapter sleeve cannula, the stop member configured to engage the proximal end of the odontoid guide cannula and prevent the adapter sleeve from translating distally with respect to the odontoid guide.

15. A method for repairing an odontoid fracture in a patient through use of an odontoid guide comprising an odontoid guide cannula having a first longitudinal passage, the method comprising:
receiving bone cement within the first longitudinal passage of the odontoid guide cannula; and
delivering the bone cement through the first longitudinal passage of the odontoid guide cannula to an odontoid process of the patient.

16. The method of claim 15, further comprising a bone cement plunger and a bone cement cannula having a second longitudinal passage, the method further comprising:
aligning a distal end of the bone cement cannula with a proximal end of the odontoid guide cannula;
inserting the distal end of the bone cement cannula into the first longitudinal passage of the odontoid guide cannula until the distal end of the bone cement cannula is received within a bone tunnel formed in the odontoid process of the patient;
aligning a distal end of the bone cement plunger with a proximal end of the bone cement cannula;
inserting the distal end of the bone cement plunger into the second longitudinal passage of the bone cement cannula;
pushing the bone cement plunger distally within the second longitudinal passage of the bone cement cannula;
ejecting bone cement from the distal end of the bone cement cannula as the bone cement plunger is pushed distally; and
filling at least a portion of the bone tunnel formed in the odontoid process with bone cement that is ejected from the distal end of the bone cement cannula.

17. The method of claim 16, further comprising:
aligning a distal end of an adapter sleeve with a proximal end of the odontoid guide cannula;
inserting the adapter sleeve into the first longitudinal passage of the odontoid guide cannula until a stop member of the adapter sleeve engages the proximal end of the odontoid guide cannula and prevents the adapter sleeve from further distal translation with respect to the odontoid guide;
aligning the distal end of the bone cement cannula with a proximal end of the adapter sleeve; and
inserting the distal end of the bone cement cannula into a third longitudinal passage formed in the adapter sleeve until the distal end of the bone cement cannula is received within the bone tunnel formed in the odontoid process of the patient.

18. The method of claim 16, wherein ejecting bone cement from the distal end of the bone cement cannula further comprises:
applying a first force, directed proximally, to a bone cement retainer handle coupled to the proximal end of the bone cement cannula; and
applying a second force, directed distally in opposition to the first force, to a bone cement plunger handle coupled to the proximal end of the bone cement plunger to move the bone cement plunger distally with respect to the bone cement cannula and eject the bone cement from the distal end of the bone cement cannula.

19. The method of claim 16, wherein the odontoid guide comprises a first spike and a second spike projecting from the distal end of the odontoid guide cannula, the method further comprising:
placing the distal end of the odontoid guide cannula proximate a first vertebra of the patient; and
coupling the odontoid guide to the first vertebra by:
applying one or more impact forces to the proximal end of the odontoid guide cannula; and
driving the first and second spikes into the first vertebra to couple the odontoid guide to the first vertebra.

20. The method of claim 19, further comprising:
applying one or more first manipulation forces to the proximal end of the odontoid guide cannula;
aligning the first vertebra relative to a second vertebra of the patient via the one or more first manipulation forces applied to the proximal end of the odontoid guide cannula;
applying one or more second manipulation forces to the proximal end of the odontoid guide cannula; and
aligning the second vertebra relative to the odontoid process located proximate the second vertebra via the one or more second manipulation forces applied to the proximal end of the odontoid guide cannula.
